SEQUENCE LISTING (6 sequences)

SEQ ID NO: 1 - DNA, 64 bp, Artificial sequence
  Synthetic primer
cgcccgccgccgcccgccgcccgtcccgccatatagtcacattttcattatttttattataagg

SEQ ID NO: 2 - DNA, 25 bp, Artificial sequence
  Synthetic primer
aattagctgtatcgtcaaggcactc

SEQ ID NO: 3 - DNA, 50 bp, Artificial sequence
  Synthetic primer
cgcccgccgccgcccgccgcccgtcccgccgctcatcgtcatcctcggca

SEQ ID NO: 4 - DNA, 24 bp, Artificial sequence
  Synthetic primer
aagtagcgaagcgagcaggactgg

SEQ ID NO: 5 - DNA, 209 bp, Homo sapiens
aggcactggtcagaatgaagtgaatggcacacaggacaagtccagacccaggaaggtccagtaacatgggagaagaacggaaggagttctaaaattcagggctcccttgggctcccctgtttaaaaatgtaggttttattattatatttcattgttaacaaaagtccatgagatctgtggaggataaagggggagctgtattttccatt

SEQ ID NO: 6 - DNA, 209 bp, Homo sapiens
aggcactggtcagaatgaagtgaatggcacacaggacaagtccagacccaggaaggtccagtaacatgggagaagaacggaaggagttctaaaattcagggctcccttgggctcccctgtttaaaaatgtaggttttattattatatttcattgttaacaaaagtccgtgagatctgtggaggataaagggggagctgtattttccatt

(19) United States
(12) Patent Application Publication
(10) Pub. No.: US 2003/0175723 A1
(43) Pub. Date: Sep. 18, 2003

(54) Methods and compositions for mutation analysis

(76) Inventors: Douglas T. Gjerde, Saratoga, CA (US); Christopher P. Hanna, Greenfield, MA (US); Paul D. Taylor, Gilroy, CA (US); Joanne Walter, Congleton (GB); Michael Daniels, Mrodlewich (GB); Carol Griffiths, Northwich (GB); Robert M. Haefele, Campbell, CA (US)

Correspondence Address: Keith Johnson, Esq., Transgenomic, Inc., 12325 Emmett Street, Omaha, NE, 68164, US

(21) Appl. No.: 10/126,848

(22) Filed: Apr. 19, 2002

Related U.S. Application Data
(63) Continuation-in-part of application No. 09/698,938, filed on Oct. 26, 2000, now Pat. No. 6,455,692.
Application No. 09/698,938 is a continuation of application No. 09/129,105, filed on Aug. 4, 1998, now Pat. No. 6,287,822.
(60) Provisional application No. 60/285,053, filed on Apr. 19, 2001.
(60) Provisional application No. 60/317,545, filed on Sep. 5, 2001.
(60) Provisional application No. 60/335,909, filed on Nov. 1, 2001.
(60) Provisional application No. 60/334,671, filed on Oct. 31, 2001.

(51) Int. Cl. C12Q 1/68; C07H 21/04; C12P 19/34

(52) U.S. Cl. 435/6; 435/91.2; 536/24.3

(57) ABSTRACT

In one aspect, a method for DNA mutation detection including the steps of PCR amplification using preferably Pho DNA polymerase, hybridization, and analysis by denaturing high performance liquid chromatography (DHPLC), the method preferably utilizing a PCR buffer and other solutions that are compatible with DHPLC analysis. In other aspects, compositions and kits including a proofreading DNA polymerase, preferably Pho DNA polymerase, and a DHPLC compatible PCR buffer are provided.

### CROSS-REFERENCE TO RELATED CO-PENDING APPLICATIONS

This application is a continuation-in-part of U.S. patent application Ser. No. 09/698,938 filed Oct. 26, 2000, which is a continuation of Ser. No. 09/129,105, filed Aug. 4, 1998 (now U.S. Pat. No. 6,287,822). This application is a regular U.S. patent application under 35 U.S.C. §111 (a) and 37 U.S.C. §1.53(b) and claims priority from the following co-pending, commonly assigned provisional applications, each filed under 35 U.S.C. §111 (b):

60/285,053 Apr. 19, 2001

60/317,545 Sep. 5, 2001

60/335,909 Nov. 1, 2001

60/334,671 Oct. 31, 2001

### FIELD OF THE INVENTION

The present invention concerns improvements in the detection of mutations in nucleic acids. The invention concerns methods, compositions, and kits for mutation analysis using denaturing high performance liquid chromatography (DHPLC). In particular, the invention concerns DNA polymerase enzymes, and PCR buffers used in preparing samples for mutation analysis by DHPLC.

### BACKGROUND OF THE INVENTION

The ability to detect mutations in double stranded polynucleotides, and especially in DNA fragments, is of great importance in medicine, as well as in the physical and social sciences. The Human Genome Project is providing an enormous amount of genetic information which is setting new criteria for evaluating the links between mutations and human disorders (Guyer et al., Proc. Natl. Acad. Sci. U.S.A 92:10841 (1995)). The ultimate source of disease, for example, is described by genetic code that differs from wild type (Cotton, TIG 13:43 (1997)). Understanding the genetic basis of disease can be the starting point for a cure. Similarly, determination of differences in genetic code can provide powerful and perhaps definitive insights into the study of evolution and populations (Cooper, et. al., Human Genetics vol. 69:201 (1985)). Understanding these and other issues related to genetic coding is based on the ability to identify anomalies, i.e., mutations, in a DNA fragment relative to the wild type. A need exists, therefore, for a methodology to detect mutations in an accurate, reproducible and reliable manner.

DNA molecules are polymers comprising sub-units called deoxynucleotides. The four deoxynucleotides found in DNA comprise a common cyclic sugar, deoxyribose, which is covalently bonded to any of the four bases, adenine (a purine), guanine (a purine), cytosine (a pyrimidine), and thymine (a pyrimidine), hereinbelow referred to as A, G, C, and T respectively. A phosphate group links a 3′-hydroxyl of one deoxynucleotide with the 5′-hydroxyl of another deoxynucleotide to form a polymeric chain. In double stranded DNA, two strands are held together in a helical structure by hydrogen bonds between, what are called, complementary bases. The complementarity of bases is determined by their chemical structures. In double stranded DNA, each A pairs with a T and each G pairs with a C, i.e., a purine pairs with a pyrimidine. Ideally, DNA is replicated in exact copies by DNA polymerases during cell division in the human body or in other living organisms. DNA strands can also be replicated in vitro by means of the Polymerase Chain Reaction (PCR).

Sometimes, exact replication fails and an incorrect base pairing occurs, which after further replication of the new strand results in double stranded DNA offspring containing a heritable difference in the base sequence from that of the parent. Such heritable changes in base pair sequence are called mutations.

In the present invention, double stranded DNA is referred to as a duplex. When the base sequence of one strand is entirely complementary to base sequence of the other strand, the duplex is called a homoduplex. When a duplex contains at least one base pair which is not complementary, the duplex is called a heteroduplex. A heteroduplex can be formed during DNA replication when an error is made by a DNA polymerase enzyme and a non-complementary base is added to a polynucleotide chain being replicated. A heteroduplex can also be formed during repair of a DNA lesion. Further replications of a heteroduplex will, ideally, produce homoduplexes which are heterozygous, i.e., these homoduplexes will have an altered sequence compared to the original parent DNA strand. When the parent DNA has the sequence which predominates in a natural population it is generally called the “wild type.”

Many different types of DNA mutations are known. Examples of DNA mutations include, but are not limited to, “point mutation” or “single base pair mutations” wherein an incorrect base pairing occurs. The most common point mutations comprise “transitions” wherein one purine or pyrimidine base is replaced for another and “transversions” wherein a purine is substituted for a pyrimidine (and visa versa). Point mutations also comprise mutations wherein a base is added or deleted from a DNA chain. Such “insertions” or “deletions” are also known as “frameshift mutations”. Although they occur with less frequency than point mutations, larger mutations affecting multiple base pairs can also occur and may be important. A more detailed discussion of mutations can be found in U.S. Pat. No. 5,459,039 to Modrich (1995), and U.S. Pat. No. 5,698,400 to Cofton (1997). These references and the references contained therein are incorporated in their entireties herein.

The sequence of base pairs in DNA codes for the production of proteins. In particular, a DNA sequence in the exon portion of a DNA chain codes for a corresponding amino acid sequence in a protein. Therefore, a mutation in a DNA sequence may result in an alteration in the amino acid sequence of a protein. Such an alteration in the amino acid sequence may be completely benign or may inactivate a protein or alter its function to be life threatening or fatal. Intronic mutations at splice sites may also be causative of disease (e.g.β-thalassemia). Mutation detection in an intron section may be important by causing altered splicing of mRNA transcribed from the DNA, and may be useful, for example, in a forensic investigation.

Detection of mutations is, therefore, of great interest and importance in diagnosing diseases, understanding the origins of disease and the development of potential treatments. Detection of mutations and identification of similarities or differences in DNA samples is also of critical importance in increasing the world food supply by developing diseases resistant and/or higher yielding crop strains, in forensic science, in the study of evolution and populations, and in scientific research in general (Guyer et al., Proc. Natl. Acad. Sci. U.S.A 92:10841 (1995); Cotton, TIG 13:43 (1997)). These references and the references contained therein are incorporated in their entireties herein.

Alterations in a DNA sequence which are benign or have no negative consequences are sometimes called “polymorphisms”. In the present invention, any alterations in the DNA sequence, whether they have negative consequences or not, are called “mutations”. It is to be understood that the method of this invention has the capability to detect mutations regardless of biological effect or lack thereof. For the sake of simplicity, the term “mutation” will be used throughout to mean an alteration in the base sequence of a DNA strand compared to a reference strand. It is to be understood that in the context of this invention, the term “mutation” includes the term “polymorphism” or any other similar or equivalent term of art.

Analysis of DNA samples has historically been done using gel electrophoresis. Capillary electrophoresis has been used to separate and analyze mixtures of DNA. However, these methods cannot distinguish point mutations from homoduplexes having the same base pair length.

Recently, a chromatographic method called ion-pair reverse-phase high pressure liquid chromatography (IP-RP-HPLC), also referred to as Matched Ion Polynucleotide Chromatography (MIPC), was introduced to effectively separate mixtures of double stranded polynucleotides, in general and DNA, in particular, wherein the separations are based on base pair length (Huber, et al., Chromatographia 37:653 (1993); Huber, et al., Anal. Biochem. 212:351 (1993); U.S. Pat. Nos. 5,585,236; 5,772,889; 5,972,222; 5,986,085; 5,997,742; 6,017,457; 6,030,527; 6,056,877; 6,066,258; 6,210,885; and U.S. patent application Ser. No. 09/129,105 filed Aug. 4, 1998.

As the use and understanding of IP-RP-HPLC developed it became apparent that when IP-RP-HPLC analyses were carried out at a partially denaturing temperature, i.e., a temperature sufficient to denature a heteroduplex at the site of base pair mismatch, homoduplexes could be separated from heteroduplexes having the same base pair length (Hayward-Lester, et al., Genome Research 5:494 (1995); Underhill, et al., Proc. Natl. Acad. Sci. U.S.A 93:193 (1996); Doris, et al., DHPLC Workshop, Stanford University, (1997)). These references and the references contained therein are incorporated herein in their entireties. Thus, the use of denaturing high performance liquid chromatography (DHPLC) was applied to mutation detection (Underhill, et al., Genome Research 7:996 (1997); Liu, et al., Nucleic Acid Res., 26;1396 (1998)).

These chromatographic methods are generally used to detect whether or not a mutation exists in a test DNA fragment. In a typical experiment, a test nucleic acid fragment is hybridized with a wild type fragment and analyzed by DHPLC. If the test fragment contains a mutation, then the hybridization product ideally includes both homoduplex and heteroduplex molecules. If no mutation is present, then the hybridization only produces homoduplex wild type molecules. The elution profile of the hybridized test fragment can be compared to a control in which a wild type fragment is hybridized to another wild type fragment. Any change in the elution profile (such as the appearance of new peaks or shoulders) between the hybridized test fragment and the control is assumed to be due to a mutation in the test fragment.

Single nucleotide polymorphisms (SNPs) are thought to be ideally suited as genetic markers for establishing genetic linkage and as indicators of genetic diseases (Landegre et al. Science 242:229-237 (1988)). In some cases a single SNP is responsible for a genetic disease. According to estimates the human genome may contain over 3 million SNPs. Due to their propensity they lend themselves to very high resolution genotyping. The SNP consortium, a joint effort of 10 major pharmaceutical companies, has announced the development of 300,000 SNP markers and their placement in the public domain by mid 2001.

The efficiency of DHPLC for detection of novel mutations (frequently termed scanning) has been quantified by several authors. Results ranged from 87% detection when a single-temperature analysis was used without any amplicon design (Cargill, et al. Nature Genet. 22:231-238 (1999)) to 100% detection in a blinded study of many polymorphisms within a single, well-behaved amplicon (O'Donovan et al., Genomics 52:44-49 (1998)). Comparisons with single-strand conformation polymorphism (SSCP) (Choy et al., Ann. Hum. Genet. 63:383-391 (1999); Gross et al., Hum. Genet. 105:72-78 (1999); Dobson-Stone et al., Eur. J. Hum. Genet. 8:24-32. (2000)) and denaturing gradient gel electrophoresis (DGGE) (Skopek et al., Mutat. Res. 430:13-21 (1999)) have shown DHPLC to have a superior detection rate, whereas most recently DHPLC has been shown to detect mutations reliably in BRCA1 and BRCA2 (Wagner et al., Genomics 62:369-376 (1999)).

A need exists to identify and optimize all the aspects of the DHPLC methodology in order to minimize artifacts and remove ambiguity from the analysis of samples containing putative mutations.

The ability of DHPLC to detect mutations may be less than 100% in some cases. There is a need for methods, compositions, and devices for improving the ability of DHPLC to detect mutations.

### SUMMARY OF THE INVENTION

In one aspect, the invention provides a method for mutation detection of a double stranded DNA fragment by DHPLC (denaturing high performance liquid chromatography), the double stranded DNA fragment corresponding to a wild type double stranded DNA fragment having a known nucleotide sequence. The method includes (a) amplifying a section of the double stranded DNA fragment by PCR using a set of primers which flank the ends of the section, wherein the PCR is conducted with Pho DNA polymerase; (b) hybridizing the amplification product of step (a) with wild type double stranded DNA corresponding to the section, whereby a mixture comprising one or more heteroduplexes is formed if the section includes a mutation; and (c) analyzing the product of step (b) by denaturing high performance liquid chromatography. The section being amplified can be indicative of a disease state.

In another aspect, the invention concerns a method for mutation detection of a double stranded DNA fragment by denaturing high performance liquid chromatography, the double stranded DNA fragment corresponding to a wild type double stranded DNA fragment having a known nucleotide sequence, in which the method includes the steps of: (a) in a PCR mixture, amplifying a section of the double stranded DNA fragment by PCR using a set of primers which flank the ends of the section, wherein the PCR is conducted with a proofreading DNA polymerase; (b) hybridizing the amplification product of step (a) with wild type double stranded DNA corresponding to the section, whereby a mixture comprising one or more heteroduplexes is formed if the section includes a mutation; and (c) analyzing the product of step (b) by denaturing high performance liquid chromatography, wherein the PCR is conducted in a PCR buffer, wherein the PCR buffer is characterized by having a DHPLC Incompatibility Index no greater than 0.1, preferably no greater than 0.05, and more preferably no greater than 0.01. The PCR buffer can include one or more non-ionic detergents having a total concentration no greater than 0.01% volume/total volume of the PCR buffer. When the PCR is conducted in a PCR mixture, the PCR buffer can include a non-ionic detergent having a concentration no greater than 0.01% volume/total volume of the total reaction mixture. The PCR buffer preferably is substantially free from substances that can interfere with DHPLC analysis. The substances include BSA, metal ions, quanidinium, and formamide. The preferred PCR mixture is characterized by having a DHPLC Incompatibility Index no greater than 0.05, and more preferably no greater than 0.01. In certain embodiments, the detergent is present in the PCR mixture at a concentration no greater than 0.09%, preferably no greater than 0.05%, and more preferably no greater than 0.01% volume/total volume of the PCR mixture. An example of a suitable detergent is TRITON X-100 (t-octylphenoxypolyethoxyethanol). The polymerase is preferably Pho polymerase. In other embodiment, the proofreading DNA polymerase can be Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT, DEEPVENT, PFUTurbo, AmpliTaq, or a combination thereof. The polymerase can be an active mutant, variant or derivative of a proofreading DNA polymerase.

In yet another aspect, there is provided a method for preparing a sample of double stranded DNA fragment for mutation detection by denaturing high performance liquid chromatography, the double stranded DNA fragment corresponding to a wild type double stranded DNA fragment having a known nucleotide sequence, the method including: in a PCR mixture, amplifying a section of the double stranded DNA fragment by PCR using a set of primers which flank the ends of the section, wherein the PCR is conducted with Pho DNA polymerase, wherein the PCR is conducted in a PCR buffer, wherein the PCR buffer is characterized by having a DHPLC Incompatibility Index no greater than 0.01.

In other aspects, the invention provides a composition for use in preparing samples for analysis by DHPLC, in which the composition consists of a PCR buffer which is characterized by having a DHPLC Incompatibility Index of no greater than 0.1, preferably no greater than 0.05 and most preferably no greater than 0.01. The composition can also include a proofreading polymerase, preferably Pho DNA polymerase, and one or more non-ionic detergents present in a concentration no greater than 0.01% volume/total volume of said composition. The composition is preferably devoid of bovine serum albumin or other substances that can interfere with DHPLC analysis. An example of such a composition is a PCR mixture.

In still another aspect, there is provided a composition for use in preparing samples for analysis by DHPLC, the composition including: a proofreading polymerase, preferably Pho DNA polymerase, wherein the polymerase is stored in a storage solution, wherein a portion of the storage solution is included in a PCR mixture which also includes a PCR buffer, wherein the PCR mixture is characterized by a DHPLC Incompatibility Index of no greater than 0.05, and preferably no greater than 0.01. The storage solution can include a non-ionic detergent, such as t-octylphenoxypolyethoxyethanol at a concentration no greater than 0.5% volume/total volume of the storage solution, and preferably no greater than 0.1%. The storage solution is preferably devoid of substances, such as BSA, that can interfere with DHPLC analysis.

In yet another aspect, the invention includes a composition for use in preparing samples for analysis by denaturing high performance liquid chromatography, the composition including: a proofreading polymerase, wherein the polymerase is stored in a storage solution, wherein when the storage solution is characterized by having a DHPLC Incompatibility Index no greater than 0.05 and preferably no greater than 0.01.

In other aspects, the invention concerns kits for preparing a double stranded DNA for mutation detection by denaturing high performance liquid chromatography in which the kits can include one or more of: a container which contains a composition including a proofreading polymerase, preferably Pho polymerase, and which contains one or more non-ionic detergents present at a concentration no greater than 0.1%, wherein the composition is devoid of bovine serum albumin; a container which contains a mutation standard; a container which contains one or more PCR primers; a container which contains a PCR buffer, wherein the buffer is characterized by having a DHPLC Incompatibility Index no greater than 0.05 and preferably no greater than 0.01; a separation column for use in denaturing high performance liquid chromatography; a DHPLC system; a container which contains a composition comprising Pho DNA polymerase containing non-ionic detergent present in a concentration no greater than 0.1% (volume/total volume of the composition) with a container which contains a reaction buffer, wherein the reaction buffer is characterized by having a DHPLC Incompatibility Index no greater than 0.05; a container which contains a composition comprising Pho DNA polymerase, a container which contains a reaction buffer, wherein the PCR buffer contains non-ionic detergent present in a concentration no greater than 0.01% volume/volume of said buffer; a polymerase such as a proofreading DNA polymerase selected from Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT, DEEPVENT, PFUTurbo, AmpliTaq, or a mixture thereof; a polymerase which is an active mutant, variant or derivative of a proofreading DNA polymerase; a PCR mixture including one or more non-ionic detergents present at a total concentration no greater than 0.01% volume/total volume of the mixture, and wherein the PCR mixture is devoid of serum albumin; a storage solution wherein the polymerase is stored in the storage solution, wherein when the storage solution is included in a PCR mixture, the PCR mixture is characterized by having a DHPLC Incompatibility Index no greater than 0.05; a container which contains a PCR buffer, wherein the PCR buffer is characterized by having a DHPLC Incompatibility Index no greater than 0.05, wherein the buffer includes KCl, Tris, MgSO₄, and wherein the buffer includes one or more non-ionic detergents at a concentration no greater than 0.01% volume/total volume of the buffer.

In a further aspect, there is provided a method for preparing a sample of double stranded DNA fragment for mutation detection by denaturing high performance liquid chromatography, the double stranded DNA fragment corresponding to a wild type double stranded DNA fragment having a known nucleotide sequence, the method including: in a PCR mixture, amplifying a section of the double stranded DNA fragment by PCR using a set of primers which flank the ends of the section, wherein the PCR is conducted with Pho DNA polymerase, wherein the PCR is conducted in a PCR buffer, wherein the PCR buffer is characterized by having a DHPLC Incompatibility Index no greater than 0.01.

### BRIEF DESCRIPTION OF THE DRAWINGS

FIG. 1 shows a schematic representation of a hybridization to form homoduplex and heteroduplex DNA molecules and the mutation separation profile of the molecules.

FIG. 2 illustrates PCR product profiles obtained using various DNA polymerases.

FIG. 3 illustrates PCR product profiles obtained using two different DNA polymerases.

FIG. 4 shows the percentage of heteroduplex DNA produces after PCR by various DNA polymerases.

FIG. 5 illustrates a procedure for calculating the area due to heteroduplex DNA and homoduplex DNA.

FIG. 6 shows overlaid PCR product profiles obtained from multiple separate injections of the PCR product obtained from Pho polymerase.

FIG. 7 shows overlaid PCR product profiles obtained from multiple separate injections of the PCR product obtained from a non-proofreading polymerase.

FIG. 8 shows overlaid PCR product profiles obtained from multiple separate injections of the PCR product obtained from Pfu polymerase.

FIG. 9 illustrates the effect of multiple injections of a first reaction buffer on the performance of a separation column as measured by the retention time of heteroduplex DNA in a standard mixture of homoduplex and heteroduplex molecules.

FIG. 10 illustrates the effect of multiple injections of a second reaction buffer on the retention time of heteroduplex DNA in a standard mixture of homoduplex and heteroduplex molecules.

FIG. 11 is a schematic illustration showing the calculation of a DHPLC Incompatibility Index.

FIG. 12 illustrates the effect of multiple injections of a third reaction buffer on the retention time of heteroduplex DNA in a standard mixture of homoduplex and heteroduplex molecules.

FIG. 13 shows an elution profile of a mutation standard.

### DETAILED DESCRIPTION OF THE INVENTION

A reliable way to detect mutations is by hybridization of the putative mutant strand in a sample with the wild type strand (Lerman, et al., Meth. Enzymol., 155:482 (1987)). If a mutant strand is present, then, typically, two homoduplexes and two heteroduplexes will be formed as a result of the hybridization process. Hence separation of heteroduplexes from homoduplexes provides a direct method of confirming the presence or absence of mutant DNA segments in a sample. The DNA sample for mutation detection is routinely the product of a polymerase chain reaction (PCR).

The instant invention concerns methods and compositions for use during PCR amplification of DNA in preparing samples for analysis by DHPLC. In general, the present invention concerns methods, compositions, and kits and devices for preparing a sample for analysis by DHPLC. One aspect of the instant invention is based in part on the surprising discovery by Applicants that Pho DNA polymerase exhibited surprisingly improved performance as compared to a variety of other DNA polymerases. Other aspects of the invention are based on the discovery by Applicants that certain components commonly included in PCR buffers and storage solutions, such as found in commercially available PCR kits, interfere with analysis of PCR products by DHPLC.

Mutation analysis involves a DNA separation process and can be performed by a variety of liquid chromatographic separation methods. Examples of suitable liquid chromatographic methods include IP-RP-HPLC and ion exchange chromatography where these are performed under partially denaturing conditions. The use of ion exchange chromatography is disclosed in U.S. patent application Ser. No. 091756,070 filed Jan. 6, 2001 and in PCT/US00/28441 filed Oct. 12, 2000. For purposes of clarity and not by way of limitation, DHPLC is described herein.

The term “nucleic acids”, as used herein, refers to either DNA or RNA. It includes plasmids, infectious polymers of DNA and/or RNA, nonfunctional DNA or RNA, chromosomal DNA or RNA and DNA or RNA synthesized in vitro (such as by the polymerase chain reaction). “Nucleic acid sequence” or “polynucleotide sequence” refers to a single- or double-stranded polymer of deoxyribonucleotide or ribonucleotide bases read from the 5′ to the 3′ end.

The term “DNA molecule” as used herein refers to DNA molecules in any form, including naturally occurring, recombinant, or synthetic DNA molecules. The term includes plasmids, bacterial and viral DNA as well as chromosomal DNA. The term encompasses DNA fragments produced by cell lysis or subsequent manipulation of DNA molecules. Unless specified otherwise, the left hand end of single-stranded DNA sequences is the 5′ end.

The term “complementary” as used herein includes reference to a relationship between two nucleic acid sequences. One nucleic acid sequence is complementary to a second nucleic acid sequence if it is capable of forming a duplex with the second nucleic acid, wherein each residue of the duplex forms a guanosine-cytidine (G-C) or adenosine-thymidine (A-T) basepair or an equivalent basepair. Equivalent basepairs can include nucleoside or nucleotide analogues other than guanosine, cytidine, adenosine, or thymidine, which are capable of being incorporated into a nucleic acid by a DNA or RNA polymerase on a DNA template. A complementary DNA sequence can be predicted from a known sequence by the normal basepairing rules of the DNA double helix (see Watson J. D., et al. (1987) Molecular Biology of the Gene, Fourth Edition, Benjamin Cummings Publishing Company, Menlo Park, Calif., pp. 65-93). Complementary nucleic acids may be of different sizes. For example, a smaller nucleic acid may be complementary to a portion of a larger nucleic acid.

The terms “purified DNA” or “purified DNA molecule,” as used herein, include reference to DNA that is not contaminated by other biological macromolecules, such as RNA or proteins, or by cellular metabolites. Purified DNA contains less than 5% contamination (by weight) from protein, other cellular nucleic acids and cellular metabolites. The terms “unpurified DNA” or “unpurified DNA molecules” refer to preparations of DNA that have greater than 5% contamination from other cellular nucleic acids, cellular proteins and cellular metabolites. Unpurified DNA may be obtained by using a single purification step, such as precipitation with ethanol combined with either LiCI or polyethylene glycol. The term “crude cell lysate preparation” or “crude cell lysate” or “crude lysate” refers to an unpurified DNA preparation where cells or viral particles have been lysed but where there has been no further purification of the DNA.

Depending on the conditions, ion-pair reverse-phase high performance liquid chromatography (IP-RP-HPLC) separates double stranded polynucleotides by size or by base pair sequence and is therefore a preferred separation technology for detecting the presence of particular fragments of DNA of interest. IP-RP-HPLC is also referred to in the art as “Matched Ion Polynucleotide Chromatography” (MIPC).

The term “chromatographic elution profile” as used herein is defined to include the data generated by the IP-RP-HPLC method when this method is used to separate double stranded DNA fragments. The chromatographic profile can be in the form of a visual display, a printed representation of the data or the original data stream.

IP-RP-HPLC as used herein includes a chromatographic process for separating single and double stranded polynucleotides using non-polar separation media, wherein the process uses a counter ion agent, and an organic solvent to release the polynucleotides from the separation media. IP-RP-HPLC separations can be completed in less than 10 minutes, and frequently in less than 5 minutes. IP-RP-HPLC systems (e.g., the WAVE® DNA Fragment Analysis System, Transgenomic, Inc. San Jose, Calif.) are preferably equipped with computer controlled ovens which enclose the columns. Mutation detection at the temperature required for partial denaturation (melting) of the DNA at the site of mutation can therefore be easily performed. The system used for IP-RP-HPLC separations is rugged and provides reproducible results. It is preferably computer controlled and the entire analysis of multiple samples can be automated. The system preferably offers automated sample injection, data collection, choice of predetermined eluting solvent composition based on the size of the fragments to be separated, and column temperature selection based on the base pair sequence of the fragments being analyzed. The separated mixture components can be displayed either in a gel format as a linear array of bands or as an array of peaks. The display can be stored in a computer storage device. The display can be expanded and the detection threshold can be adjusted to optimize the product profile display. The reaction profile can be displayed in real time or retrieved from the storage device for display at a later time. A mutation separation profile, a genotyping profile, or any other chromatographic separation profile display can be viewed on a video display screen or as hard copy printed by a printer.

A “homoduplex” is defined herein to include a double stranded DNA fragment wherein the bases in each strand are complementary relative to their counterpart bases in the other strand.

A “heteroduplex” is defined herein to include a double stranded DNA fragment wherein at least one base in each strand is not complementary to at least one counterpart base in the other strand. Since at least one base pair in a heteroduplex is not complementary, it takes less energy to separate the bases at that site compared to its fully complementary base pair analog in a homoduplex. This results in the lower melting temperature at the site of a mismatched base of a heteroduplex compared to a homoduplex. A heteroduplex can be formed by annealing of two nearly complementary sequences.

The term “hybridization” refers to a process of heating and cooling a double stranded DNA (dsDNA) sample, e.g., heating to 95° C. followed by slow cooling. The heating process causes the DNA strands to denature. Upon cooling, the strands re-combine, or anneal, into duplexes.

When mixtures of DNA fragments are mixed with an ion pairing agent and applied to a reverse phase separation column, they are separated by size, the smaller fragments eluting from the column first. However, when IP-RP-HPLC is performed at an elevated temperature which is sufficient to denature that portion of a DNA fragment domain which contains a heteromutant site, then heteroduplexes separate from homoduplexes. IP-RP-HPLC, when performed at a temperature which is sufficient to partially denature a heteroduplex, is referred to as DHPLC. DHPLC is also referred to in the art as “Denaturing Matched Ion Polynucleotide Chromatography” (DMIPC).

In the operation of the DHPLC method, the determination of a mutation is preferably made by hybridizing the homozygous sample with the known wild type fragment and performing a DHPLC analysis at a partially denaturing temperature. If the sample contained only wild type fragments then a single peak would be seen in the DHPLC analysis since no heteroduplexes could be formed. In the operation of the DHPLC method, the determination of a mutation can be made by hybridizing the homozygous sample with the corresponding wild type fragment and performing a DHPLC analysis. If the sample contained only wild type fragments then a single peak would be seen in the DHPLC analysis since no heteroduplexes could be formed. If the sample contained homozygous mutant fragments or was heterozygous for the mutation, then analysis by DHPLC can be used to detect the separation of homoduplexes and heteroduplexes.

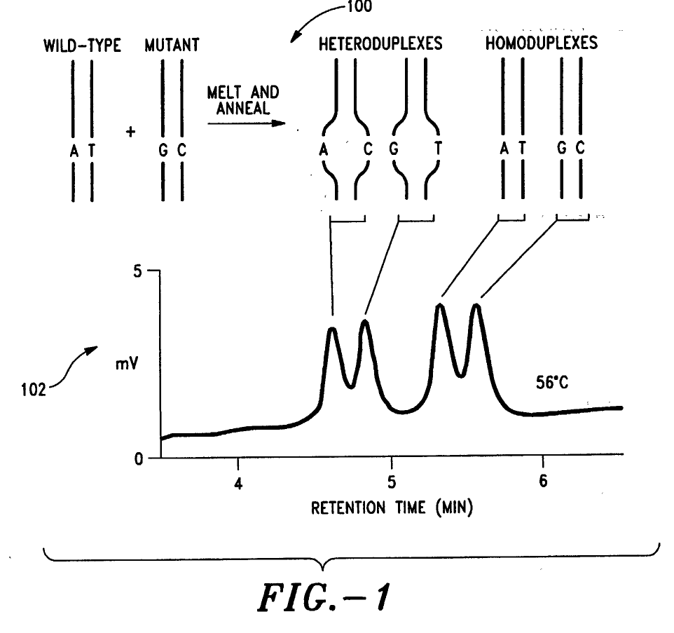

The term “mutation separation profile” is defined herein to include a DHPLC separation chromatogram which shows the separation of heteroduplexes from homoduplexes. Such separation profiles are characteristic of samples which contain mutations or polymorphisms and have been hybridized prior to being separated by DHPLC. The DHPLC separation chromatogram 102 shown in FIG. 1 exemplifies a mutation separation profile as defined herein. “Mutation standards” are defined herein to include mixtures of DNA species that when hybridized and analyzed by DHPLC, produce previously characterized mutation separation profiles which can be used to evaluate the performance of the chromatography system. Mutation standards can be obtained commercially (e.g. a WAVE® System Low Range Mutation Standard, part no. 560077, GCH338 Mutation Standard (part no. 700215), and HTMS219 Mutation Standard (part no. 700220) are available from Transgenomic, Inc. and a 209 bp mutation standard is also available from Varian, Inc. The 209 base pair mutation standard comprises a 209-bp fragment from the human Y chromosome locus DYS217 (GenBank accession number S76940)).

Analysis of a 209 bp Mutation Standard (Transgenomic) is illustrated in FIG. 1. Prior to injection of the mixture onto the separation column, the mutation standard is preferably hybridized as shown in the scheme 100. The hybridization process created two homoduplexes and two heteroduplexes. As shown in the mutation separation profile 102, the hybridization product was separated using DHPLC. The two lower retention time peaks represent the two heteroduplexes and the two higher retention time peaks represent the two homoduplexes. The two homoduplexes separate because the A-T base pair denatures at a lower temperature than the C-G base pair. Without wishing to be bound by theory, the results are consistent with a greater degree of denaturation in one duplex and/or a difference in the polarity of one partially denatured heteroduplex compared to the other, resulting in a difference in retention time on the reverse-phase separation column.

Detection of unknown mutations requires a highly sensitive, reproducible and accurate analytical method. The design of polymerase chain reaction (PCR) primers used to amplify DNA samples which are to be analyzed for the presence of mutations is an important factor contributing to accuracy, sensitivity and reliability of mutation detection. The design of primers specifically for the purpose of enhancing and optimizing mutation detection by DHPLC is disclosed in U.S. patent application Ser. No. 10/033,104 filed Oct. 29, 2001, U.S. Pat. No. 6,287,822, PCT publication WO9907899, PCT publication PCTUS01/45676 filed Oct. 29, 2001, by Xiao et al. (Human Mutation 17:439-474 (2001) and by Kuklin etal., (Genet. Test. 1:201-206 (1998).

Stationary phases for carrying out the separation include reverse-phase supports composed of alkylated base materials, such as silica, polyacrylamide, alumina, zirconia, polystyrene, and styrene-divinyl copolymers. Styrene-divinyl copolymer base materials include copolymers composed of i) a monomer of styrene such as styrene, alkyl-substituted styrenes, α-methylstyrene, or alkyl substituted α-methylstyrenes and ii) a divinyl monomer such as divinylbenzene or divinylbutadiene. In one embodiment, the surface of the base material is alkylated with hydrocarbon chains containing from about 4-18 carbon atoms. In another embodiment, the stationary support is composed of beads from about 1-100 microns in size.

Examples of suitable separation media are described in the following U.S. patents and patent applications: U.S. Pat. Nos. 6,056,877; 6,066,258; 5,453,185; 5,334,310; U.S. patent application Ser. No. 09/493,734 filed Jan. 28, 2000; U.S. patent application Ser. No. 09/562,069 filed May 1, 2000; and in the following PCT applications: WO98/48914; WO98/48913; PCT/US98/08388; PCT/US00/11795.

An example of a suitable column based on a polymeric stationary support is the DNASep® column (Transgenomic). An example of a suitable column based on a silica stationary support is the Microsorb Analytical column (Varian and Rainin).

Monolithic columns, including capillary columns, can also be used, such as disclosed in U.S. Pat. No. 6,238,565; U.S. patent application Ser. No. 09/562,069 filed May 1, 2000; the PCT application WO00/15778; and by Huber et al (Anal. Chem. 71:3730-3739 (1999)).

The length and diameter of the separation column, as well as the system mobile phase pressure and temperature, and other parameters, can be varied as is known in the art.

Size-based separation of DNA fragments can also be performed using batch methods and devices as disclosed in U.S. Pat. Nos. 6,265,168; 5,972,222; and 5,986,085.

In DHPLC, the mobile phase contains an ion-pairing agent (i.e. a counter ion agent) and an organic solvent. Ion-pairing agents for use in the method include lower primary, secondary and tertiary amines, lower trialkylammonium salts such as triethylammonium acetate and lower quaternary ammonium salts. Typically, the ion-pairing reagent is present at a concentration between about 0.05 and 1.0 molar. Organic solvents for use in the method include solvents such as methanol, ethanol, 2-propanol, acetonitrile, and ethyl acetate.

In one embodiment, the mobile phase for carrying out the separation of the present invention contains less than about 40% by volume of an organic solvent and greater than about 60% by volume of an aqueous solution of the ion-pairing agent. In a preferred embodiment, elution is carried out using a binary gradient system.

At least partial denaturation of heteroduplex molecules can be carried out several ways including the following. Temperatures for carrying out the separation method of the invention are typically between about 40° and 70° C., preferably between about 55°-65° C. In a preferred embodiment, the separation is carried out at 56° C. Alternatively, in carrying out a separation of GC-rich heteroduplex and homoduplex molecules, a higher temperature (e.g., 64° C.) is preferred.

A wide variety of liquid chromatography systems are available that can be used for conducting DHPLC. These systems typically include software for operating the chromatography components, such as pumps, heaters, mixers, fraction collection devices, injector. Examples of software for operating a chromatography apparatus include HSM Control System (Hitachi), ChemStation (Agilent), VP data system (Shimadzu), Millennium32 Software (Waters), Duo-Flow software (Bio-Rad), and ProStar Biochromatography HPLC System (Varian).

Examples of preferred liquid chromatography systems for carrying out DHPLC include the WAVE® DNA Fragment Analysis System (Transgenomic) and the Varian ProStar Helix™ System (Varian).

In carrying out DHPLC analysis, the operating temperature and the mobile phase composition can be determined by trial and error. However, these parameters are preferably obtained by using software. Computer software that can be used in carrying out DHPLC is disclosed in the following patents and patent applications: U.S. Pat. No. 6,287,822; 6,197,516; U.S. patent application Ser. no. 09/469,551 filed Dec. 22, 1999; and in WO0146687 and WO0015778. Examples of software for predicting the optimal temperature for DHPLC analysis are disclosed by Jones et al. in Clinical Chem. 45:113-1140 (1999) and in the website having the address of http://insertion.stanford.edu/melt.html. And example of a commercially available software includes WAVEMaker® software and Navigator® software (Transgenomic, Inc.).

“Non-ionic polymeric detergents” refers to surface-active agents that have no ionic charge and which can stabilize a polymerase enzyme herein at a pH range of from about 3.5 to about 9.5, preferably from 4 to 8.5.

For long-term stability, the polymerae enzyme herein can be stored in a buffer that contains one or more non-ionic polymeric detergents. The PCR buffers described herein can include one or more non-ionic detergents. Such detergents are generally those that have a molecular weight in the range of approximately 100 to 250,000, preferably about 4,000 to 200,000 daltons and stabilize the enzyme at a pH of from about 3.5 to about 9.5, preferably from about 4 to 8.5. Examples of such detergents include those specified on pages 295-298 of McCutcheon's Emulsifiers & Detergents, North American edition (1983), published by the McCutcheon Division of MC Publishing Co., 175 Rock Road, Glen Rock, N.J. (USA), the entire disclosure of which is incorporated herein by reference. Preferably, the detergents are selected from the group comprising ethoxylated fatty alcohol ethers and lauryl ethers, ethoxylated alkyl phenols, octylphenoxy polyethoxy ethanol compounds, modified oxyethylated and/or oxypropylated straight-chain alcohols, polyethylene glycol monooleate compounds, polysorbate compounds, and phenolic fatty alcohol ethers. The detergent can be selected from the group consisting of a polyoxyethylated sorbitan monolaurate, an ethoxylated nonyl phenol, ethoxylated fatty alcohol ethers, laurylethers, ethoxylated alkyl phenols, octylphenoxy polyethoxy ethanol compounds, modified oxyethylated and/or oxypropylated straight chain alcohols, polyethylene glycol monooleate compounds, polysorbate compounds, and phenolic fatty alcohol ethers or a combination thereof. The detergent can be a polyoxyethylated sorbitan monolaurate, an ethoxylated nonyl phenol or a combination thereof. More particularly preferred are Tween 20, from ICI Americas Inc., Wilmington, Del., which is a polyoxyethylated (20) sorbitan monolaurate, lconol.TM. NP-40, from BASF Wyandotte Corp. Parsippany, N.J., which is an ethoxylated alkyl phenol (nonyl), and Triton® X-100 (t-octylphenoxypolyethoxyethanol available from Sigma-Aldrich, catalogue no. T9284), Nonidet P-40, or a combination thereof.

The present invention involves nucleic acid amplification procedures, such as PCR, which involve chain elongation by a DNA polymerase. There are a variety of different PCR techniques which utilize DNA polymerase enzymes, such as Taq polymerase. See PCR Protocols: A Guide to Methods and Applications. (Innis, M, Gelfand, D., Sninsky, J. and White, T., eds.), Academic Press, San Diego (1990) for detailed description of PCR methodology. PCR is also described in detail in U.S. Pat. No. 4,683,202 to Mullis (1987); Eckert et al., The Fidelity of DNA polymerases Used In The Polymerase Chain Reactions, McPherson, Quirke, and Taylor (eds.), “PCR: A Practical Approach”, IRL Press, Oxford, Vol. 1, pp. 225-244; Andre, et. al., GENOME RESEARCH, Cold Spring Harbor Laboratory Press, pp. 843-852 (1977).

In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

Oligonucleotide primers useful in the present invention may be any oligonucleotide of two or more nucleotides in length. Preferably, PCR primers are about 15 to about 30 bases in length, and are not palindromic (self-complementary) or complementary to other primers that may be used in the reaction mixture. Oligonucleotide primers are oligonucleotides used to hybridize to a region of a target nucleic acid to facilitate the polymerization of a complementary nucleic acid. Any primer may be synthesized by a practitioner of ordinary skill in the art or may be purchased from any of a number of commercial venders (e.g., from Boehringer Mannheim Corp., Indianapolis, Ind.; New England Biolabs, Inc., Beverley, Mass.; Pharmacia LKB Biotechnology, Inc., Piscataway, N.J.). It will be recognized that the PCR primers can include covalently attached groups, such as fluorescent tags. U.S. Pat. No. 6,210,885 describes the use of such tags in mutation detection by DHPLC. It is to be understood that a vast array of primers may be useful in the present invention, including those not specifically disclosed herein, without departing from the scope or preferred embodiments thereof.

The PCR process is limited in its ability to replicate DNA strands by the specificity of the DNA polymerase used, as well as other features of the reaction. For example, the primers may bind to portions of a DNA strand which are only partially complementary. Such nonspecific primer binding will produce products with an undesired sequence. In addition, the first and second primers may also bind to complementary portions of each other, producing primer dimers. The specificity of DNA polymerases varies with the reaction conditions employed as well as with the type of enzyme used. No enzyme affords completely error-free extensions of a primer. A non-complementary base will be introduced from time to time. Such polymerase related errors produce double stranded DNA products which are not exact copies of the original DNA sample, that is, the products contain PCR induced mutations. Other PCR process variables which may influence the accuracy or fidelity of DNA replication include reaction temperature, primer annealing temperature, enzyme concentration, dNTP concentration, Mg⁺⁺ concentration, source of the polymerase and combinations thereof.

Many applications of PCR require the highest level of replication fidelity which can be achieved. In particular, the construction of genetically engineered monoclonal antibodies, analysis of T-cell receptor allelic polymorphism, the study of HIV variation in vivo and cloning of individual DNA molecules from the PCR amplified population depend upon high fidelity amplification for their success.

The term “PCR product profile” as used herein is defined to include the data generated by DHPLC as applied to the product of a PCR process. The DHPLC data can distinguish the expected product and other components of the reaction mixture from one another. These components comprise desired product(s), byproducts and reaction artifacts. The PCR product profile can be in the form of a visual display, a printed representation of the data or the original data stream.

The degree of fidelity of replication of DNA fragments by PCR depends on many factors which have long been recognized in the art. Some of these factors are interrelated in the sense that a change in the PCR product profile caused by an increase or decrease in the quantity or concentration of one factor can be offset, or even reversed by a change in a different factor. For example, an increase in the enzyme concentration may reduce the fidelity of replication, while a decrease in the reaction temperature may increase the replication fidelity. An increase in magnesium ion concentration or dNTP concentration may result in an increased rate of reaction which may have the effect of reducing PCR fidelity. A detailed discussion of the factors contributing to PCR fidelity is presented by Eckert et al., (in PCR: A Practical Approach, McPherson, Quirke, and Taylor eds., IRL Press, Oxford, Vol. 1, pp. 225-244, (1991)); and Andre, et. al., (GENOME RESEARCH, Cold Spring Harbor Laboratory Press, pp. 843-852 (1977)).

Buffering agents and salts are used in the PCR buffers and storage solutions of the present invention to provide appropriate stable pH and ionic conditions for nucleic acid synthesis, e.g., for DNA polymerase activity, and for the hybridization process. A wide variety of buffers and salt solutions and modified buffers are known in the art that may be useful in the present invention, including agents not specifically disclosed herein. Preferred buffering agents include, but are not limited to, TRIS, TRICINE, BIS-TRICINE, HEPES, MOPS, TES, TAPS, PIPES, CAPS. Preferred salt solutions include, but are not limited to solutions of; potassium acetate, potassium sulfate, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, and lithium acetate.

In a general aspect, the invention provides methods and compositions for high sensitivity mutation detection by DHPLC analysis. In one aspect, the invention involves the use of Pho polymerase for preparing DNA fragments for analysis by DHPLC. In another aspect, the invention involves testing PRC reaction buffers for compatibility for analysis DHPLC.

Samples to be analyzed for the presence or absence of mutations often contain amounts of material too small to detect. The first step in mutation detection assays is, therefore, sample amplification using the PCR process. PCR amplification comprises steps such as primer design, choice of DNA polymerase enzyme, the number of amplification cycles and concentration of reagents. Each of these steps, as well as other steps involved in the PCR process affects the purity of the amplified product. As a result, PCR induced mutations, wherein a non-complementary base is added to a template, are often formed during sample amplification. Such PCR induced mutations make mutation detection results ambiguous, since it may not be clear if a detected mutation was present in the sample or was produced during the PCR process. In contrast to the teachings in the prior art of mutation detection by DHPLC, Applicants have recognized the importance of optimizing PCR sample amplification by the use of proofreading DNA polymerases in order to minimize the formation of PCR induced mutations and ensure an accurate and unambiguous analysis of putative mutation containing samples.

One aspect of the instant invention concerns the use of Pho DNA polymerase in preparing amplifying DNA samples for analysis by DHPLC. This aspect of the invention is based in part on Applicants surprisingly discovery that Pho DNA polymerase yields lower rates of misincorporation of bases in PCR as compared to a wide variety of other polymerases.

Pho DNA polymerase is produced by the hyper-thermophilic archaebacterim, Pyrococcus horikoshii OT3 (Kawarabayasi et al. DNA Research 5:55-76 (1998)). A method for producing the enzyme is described in Japanese Patent 3,015,878. Methods for obtaining the enzyme include expression in the T7 expression system which system is described in U.S. Pat. Nos. 5,868,320; 4,952,496; 5,639,489. Another suitable expression system is described in U.S. Pat. No. 6,017,745. The recombinant polymerase protein can be purified by conventional methods. For example, purification of the recombinant polymerase can be facilitated by including histidine residues on the amino or carboxy terminus as known in the art (U.S. Pat. Nos. 5,310,63; 4,887,830; 5,047,513; and 5,284,933; and Current Protocols in Molecular Biology, Ausubel et al, eds, Supplement 24 CPMB pp. 10.11.8-1-.11.22 (1992)) which purification utilizes a Ni²⁺-NTA resin (available from Novagen (part no. 70666-5)).

Genomic DNA containing the gene for Pho polymerase was provided by Professor Bernard Connelly (University of Newcastle), and the gene was amplified and cloned into plasmid pQIS130R2. Site directed mutagenesis was performed on the plasmid to correct mutations occurring within the coding sequence. When this had been completed and confirmed by sequencing the coding sequence was put into an expression vector (pET 14b, CN Biosciences). The vector was expressed in E. Coli, and the resulting Pho polymerase was extracted.

Pho polymerase is available commercially (Optimase™ polymerase, Transgenomic).

In order to achieve the highest quality of DHPLC analysis it is preferred that PCR is carried out using a polymerase preparation that is both compatible with the DHPLC system and that has the highest possible fidelity during amplification.

Use of proofreading DNA polymerases was not recommended by Oefner et al. (Xiao et al. Human Mutation 17:439-474 (2001) and Oefner et al. Curent Protocols in Human Genetics, Supplement 19, pp. 7.10.1-7.10.12 (1998)): “Specialty low-error-rate thermostable polymerases are not necessary for amplification of single-copy genomic targets for DHPLC analysis.”

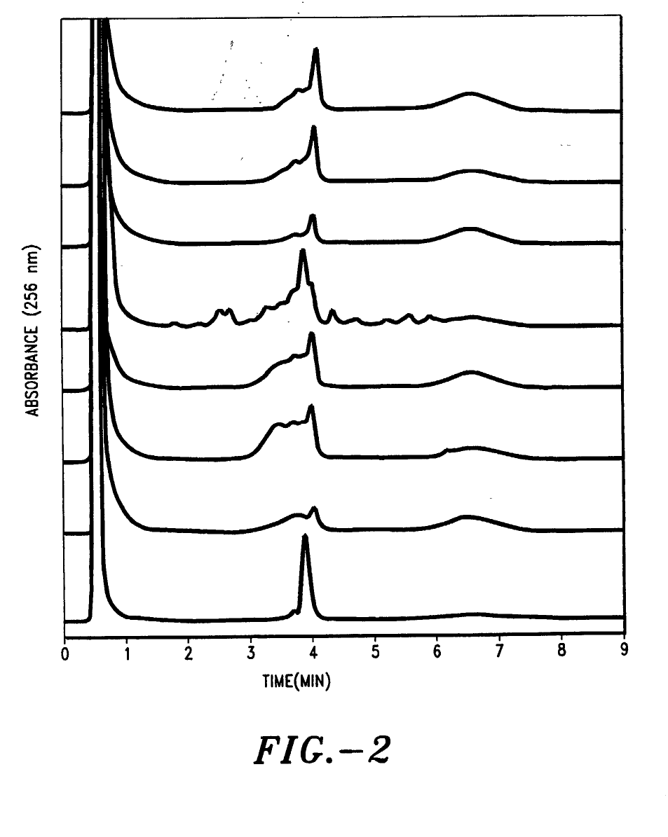

However, Applicants have discovered that PCR induced mutations can interfere with the detection of mutations using DHPLC. As described herein (EXAMPLE 2), by comparing the fidelity of PCR using a series of polymerase enzymes commonly used for PCR, Applicants surprisingly discovered that Pho polymerase gave the highest fidelity of any polymerase tested. For each DNA polymerase tested, DHPLC analysis showed the presence of two distinct forms of DNA fragment (FIG. 2). In FIG. 2, eight different polymerases were compared. The major component of each PCR product was found to be homoduplex DNA observed as a peak with a retention time of approximately 4 minutes. In addition to this major component a second peak was observed indicating the presence of heteroduplex DNA resulting from polymerase induced base misincorporations. The size of the heteroduplex peak was found to be consistent for each polymerase but varied over a considerable range between different polymerases.

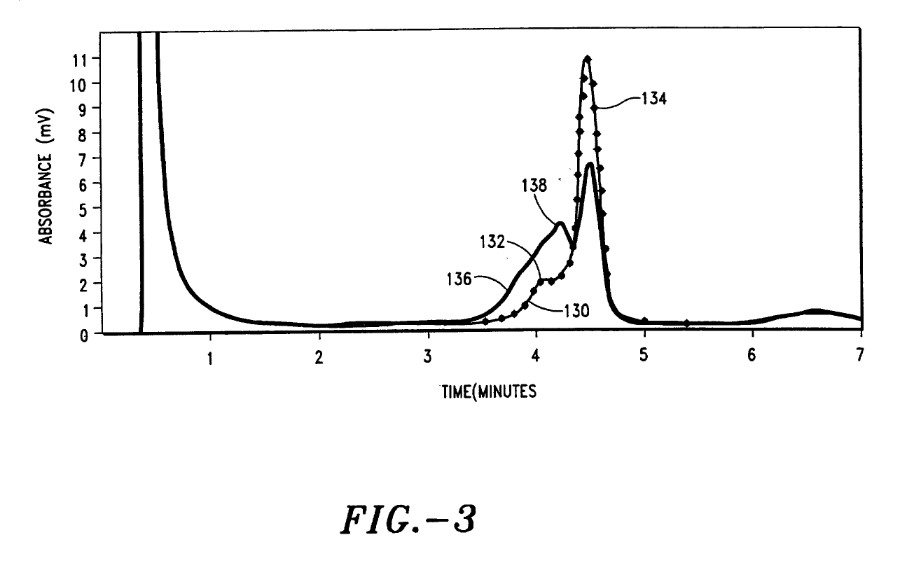

FIG. 3 provides another illustration of the effect of base misincorporations during PCR on peak profiles obtained using DHPLC. The PCR product profile 130 from analysis of amplification by Pho polymerase shows a small “bump” 132 prior to the well defined main peak 134, indicative of high quality PCR with few misincorporations. The PCR product profile 136 from analysis of amplified products of Herculase polymerase (Stratagene) shows a distinct “shoulder” 138 prior to the main peak indicating a higher level of misincorporation than for Pho. Polymerases that induce the incorporation of high numbers of errors during amplification can have a detrimental effect on data analysis.

PCR product profiles obtained for Pho polymerase 134 and Herculase 136 showed heteroduplex formation in 7.3% and 22.1% of PCR products, respectively. The affect of these misincorporations is clearly visible and at high levels of misincorporations the quality of data acquired using DHPLC can be impaired.

Results from the analysis of a variety of polymerases are presented in FIG. 4 and TABLE 1.

TABLE 1

| DNA Polymerase | Percentage of fragments containing errors |
|---|---|
| Pho | 7.43 |
| Pfu | 8.36 |
| Herculase | 9.7 |
| Gold PFUTurbo | 15.65 |
| Amplitaq Gold | 22.08 |
| Amplitaq | 27.9 |
| Pwo | 28.45 |

FIG. 4 shows the percentage of total PCR product found to form heteroduplex DNA, indicating the presence of misincorporated bases. The data in FIG. 4 and TABLE 1 correlate well with the relative error incorporation rates that have been shown for these polymerases in other studies (Cline et al. Nucleic Acids Research. 24:3546-3551 (1996); Mattila, et al. Nucleic Acids Research 19:4967-4973 (1991); Cha, et al. R. S. & Thilly, W. G. PCR Methods and Applications 3:S18-S29 (1993); Cariello, et al. Nucleic Acids Research 19:4193-4198 (1991); Keohavong, et al. Proceedings of the National Academy of Science of the USA, 86:9253-9257 (1989)), confirming that analysis of the percentage of heteroduplex fragments gives an equivalent measure of replication fidelity to those methods used elsewhere. Some variability can occur between different users and different thermocyclers illustrating the need to use high quality equipment and materials in PCR. The use of Pho polymerase produced the lowest misincorporation rate under the conditions used.

The “detection limit” is defined by the International Union of Pure and Applied Chemistry (IUPAC) and others (Thompson, Analyst 112:199-204 (1987)) as the concentration that gives rise to a signal that is equal to three times the standard deviation of the analytical blank. Thus, the lower the standard deviation of the analytical blank, the lower the limit of detection. PCR-induced mutations are the result of “PCR infidelity”, which is a well-known characteristic of PCR in general. Any and all mutation-derived mismatches within the final PCR products will give rise to heteroduplices, whether the mutation originates from the genomic DNA sequence or are introduced in the PCR. The latter instance will give rise to a significant “mutant background” signal, and can lead to an overestimation of the amount of mutant present if not taken into consideration. With respect to the minimum quantity of mutant detectable by DHPLC, and adhering the IUPAC definition of detection limits, it is the variation of the background signal itself that defines the mutation detection limits.

Applicants have determined the extent of background variation for Pho polymerase and two other polymerases, namely Taq and Pfu. The comparison between these polymerases involved performing separate amplifications of homozygous pBR322 plasmid (EXAMPLE 4 and FIGS. 6-8), so that any heteroduplices detected were the sole result of PCR-induced misincorporations, and thus the variability of these misincorporations could also be measured.

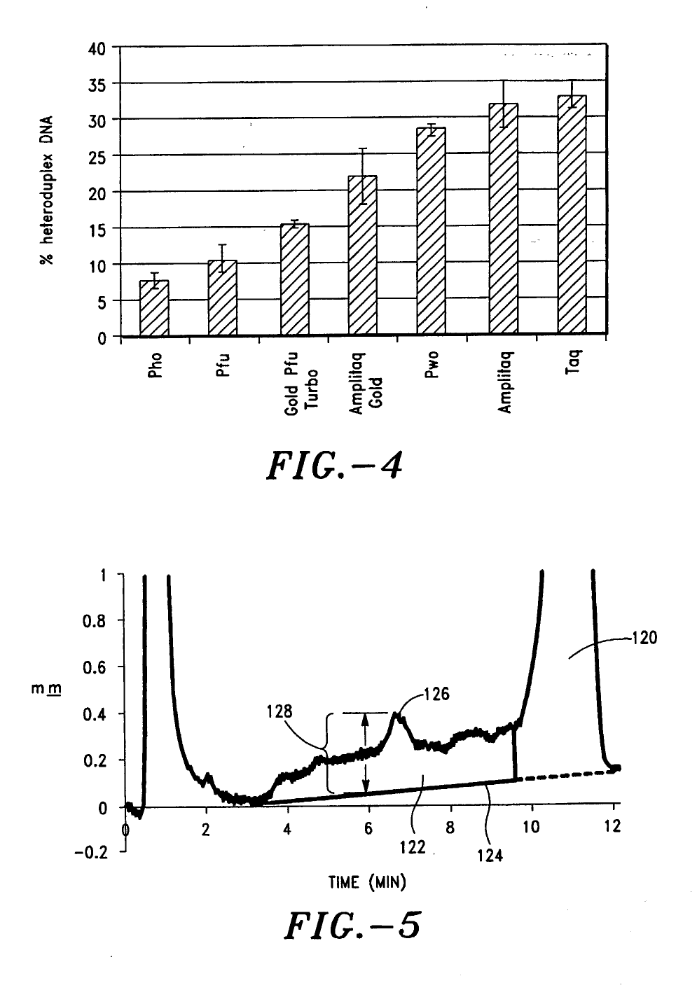

To determine the “% Heteroduplex” present in a measurement, the peak area and the peak height were measured. When measuring the peak area, the entire heteroduplex elution region was integrated. The total background heteroduplex area is proportional to the total number of PCR-induced error in the amplification. FIG. 5 illustrates the signal processing procedure for performing background signal measurements, and shows the area due to homoduplex 120, the area due to heteroduplex 122, the corrected baseline 124, the heteroduplex peak 126, and the heteroduplex peak height 128. The background peak area was determined by calculating the heteroduplex area's percentage of the total area after baseline-correction, while background peak height was determined by calculating the heteroduplex height's percentage of the total height after baseline-correction.

The instant invention is also based in part on Applicant's surprising discovery that Pho polymerase exhibits a more reproducible rate of misincorporation of bases (infidelity) as compared to other DNA polymerases. This was demonstrated in an experiment in which a sequence within pBR322 was amplified using various DNA polymerases (EXAMPLE 4), and the PCR products were analyzed using DHPLC. As shown in TABLE 2, the standard deviation of the mean for Pho polymerase was lower than for the other polymerases.

TABLE 2 shows the variation in the relative amount of misincorporations introduced by different thermostable polymerases, measured as peak area and peak height (the chromatographs for Taq, Pfu and Pho are shown in FIGS. 6-8). Six separate determinations were performed with each polymerase. Taqₚₗ represent three replicate determinations for a different Taq polymerase (“Platinum” Taq) for the amplification of the ras exon 1 alleles (EXAMPLE 3).

TABLE 2

| | Taq | Pfu | Pho | Taq_{PI} |
|---|---|---|---|---|
| Peak Area | 24.4 ± 1.7% | 15.1 ± 5.9% | 13.5 ± 2.6% | 60.4 ± 2.6% |
| (±95% C.I.): | | | | |
| Standard De- | 1.6% | 5.6% | 2.5% | 1.0% |
| iation: | | | | |
| Peak Area Det. | 4.8% | 16.8% | 7.5% | 3.0% |
| Limit: | | | | |
| Peak Height | 7.5 ± 0.6% | 3.9 ± 3.8% | 5.2 ± 1.4% | 37.6 ± 2.0% |
| (±95% C.I.): | | | | |
| Standard De- | 0.6% | 2.5% | 1.3% | 0.8% |
| iation: | | | | |
| Peak Height | 1.8% | 7.5% | 3.9% | 2.4% |
| Det. Limit: | | | | |

By comparing the mean heteroduplex “Peak area” for each polymerase, it is clear that the proofreading polymerases Pfu and Pho generate significantly fewer misincorporations than non-proofreading polymerase Taq. The 95% confidence interval around the mean background peak area for Pfu and Pho indicated that they are statistically indistinguishable from each other with respect to relative signal intensity. However, as between Pfu and Pho, Pho displayed a lower standard deviation, and therefore provided a lower peak area detection limit.

The results in TABLE 2 show that Pho polymerase gave a lower standard deviation in peak area and in peak height. Both the peak area detection limit and the peak height detection limit were significantly lower for Pho polymerase. This surprising discovery by Applicants illustrates an important advantage of using Pho polymerase in DHPLC analysis.

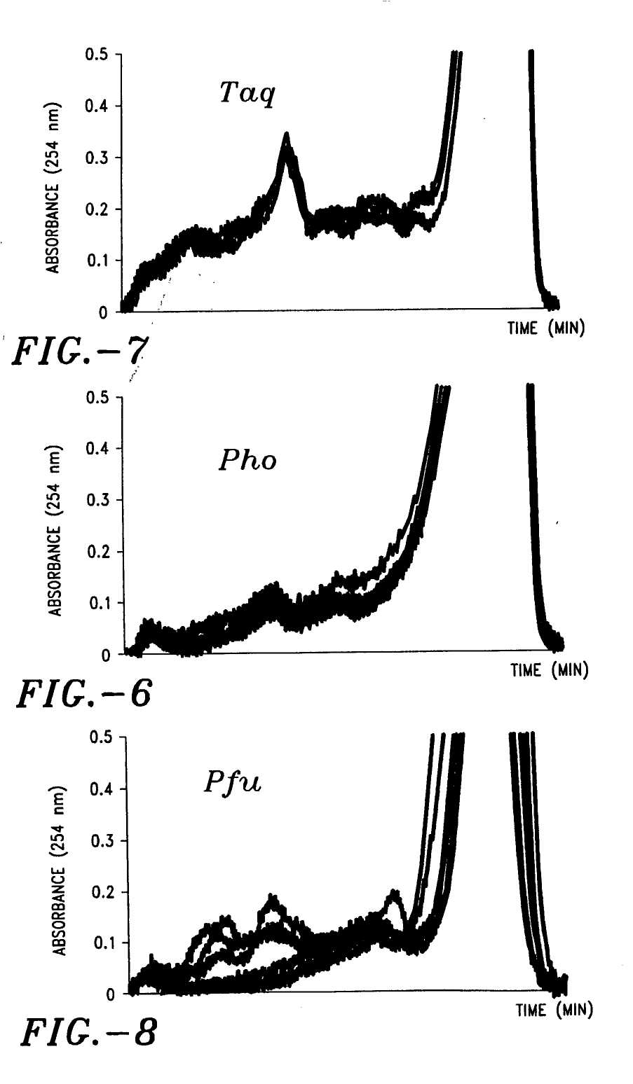

These differences in reproducibility were also demonstrated in FIGS. 6-8. The highly reproducible rate of misincorporation for Pho polymerase was apparent in a DHPLC analysis of FIG. 6. For comparison, FIG. 7 shows a set of PCR product profiles for Taq polymerase, and FIG. 8 shows a set of profiles produced from Pfu polymerase. Comparing these three figures qualitatively, it can be seen that the PCR product the profile obtained from Pfu included a higher level of PCR-induced mutations and that the reproducibility of the profile was lower than for Pho. These figures indicate that the overall heteroduplex signal shape is well conserved in the case of Taq polymerase as well as for Pho polymerase. There is considerably more variability to the heteroduplex signal shape in the case of Pfu, which indicates its having the highest degree of variability for this determination.

The present invention also concerns providing a PCR buffer, or other solution, for use in PCR that does not interfere with analysis of the PCR products by DHPLC. This aspect of the invention is based in part on the discovery by Applicants that certain components commonly included in PCR buffers and storage solutions are often incompatible with analysis of PCR products by DHPLC. Applicants have found that a number of commercially available PCR buffers and polymerase preparations, such as provided in PCR kits, are not compatible with analysis by DHPLC because of interference with the elution of DNA fragments from the separation column.

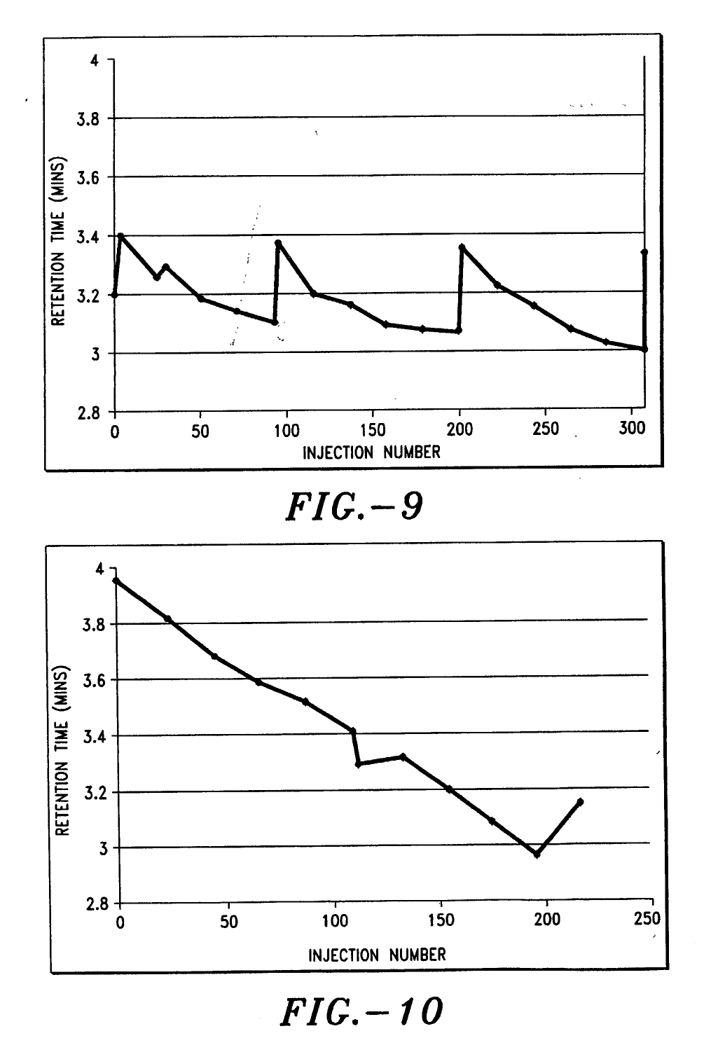

For example, FIG. 9 illustrates the effect of multiple injections of a PCR buffer obtained in the Pfu polymerase kit sold by Stratagene on the performance of a separation column as measured by the retention time of heteroduplex DNA in the 209 bp Mutation Standard mixture of homoduplex and heteroduplex molecules. The retention time of the heteroduplex peak decreased after multiple injections of the PCR buffer tested. A washing procedure was used to regenerate the separation column.

As another example, FIG. 5 illustrates the effect of multiple injections of a PCR buffer in the Herculase polymerase kit sold by Stratagene on the performance of a separation column as measured by the retention time of heteroduplex DNA in the 209 bp Mutation Standard mixture of homoduplex and heteroduplex molecules. The retention time of the heteroduplex peak decreased after multiple injections of the PCR buffer tested. A washing procedure was not effective in regenerating the separation column. This PCR buffer was determined to contain BSA.

In addition, Applicants have herein devised a method for testing PCR buffers, and other solutions that are to be used in PCR, for compatibility with analysis by DHPLC.

Applicants have found that the concentrations of the ingredients in the PCR buffer can be manipulated such that the buffer is operable during PCR and is also compatible with the separation of the PCR products using DHPLC.

Another aspect of the instant invention provides a method for quantifying the compatibility of a buffer or other solution that is to be analyzed by DHPLC. The calculation of a “DHPLC Incompatibility Index” is illustrated in FIG. 11 and described in EXAMPLE 5. Briefly, a Mutation Standard (i.e. a mixture of known homoduplex and heteroduplex fragments) is injected onto the separation column and eluted at a temperature which partially denatures at a site of mismatch and a chromatogram is recorded. The retention time of the earliest eluting heteroduplex peak in the chromatograph is obtained. After multiple injections of the solution being characterized, e.g. a PCR buffer diluted to its working concentration, the Mutation Standard is again injected, and the retention time of the first eluting heteroduplex peak is compared to the retention time of the first eluting heteroduplex peak prior to the multiple injections. The DHPLC Incompatibility Index is calculated as described in EXAMPLE 5.

Applicants have found PCR buffers or other solutions that are characterized by a DHPLC Incompatibility Index of no greater than 0.1 can be operable for use in DHPLC analysis, while values no greater than 0.05 are more preferred, and values no greater than 0.01 are most preferred.

The determination of this Index allows one to test whether a PCR buffer, or any other solution, will be compatible with the DHPLC system. It will be appreciated, that by the use of this Index, PCR buffers and other solutions can be designed to select components and component concentrations in order to minimize interference with analysis by DHPLC. For example, in use, this method can be used to test a mixture that includes a preparation of a proofreading DNA polymerase combined with a PCR buffer, but without PCR primers or template, in order to simulate the conditions present during a PCR.

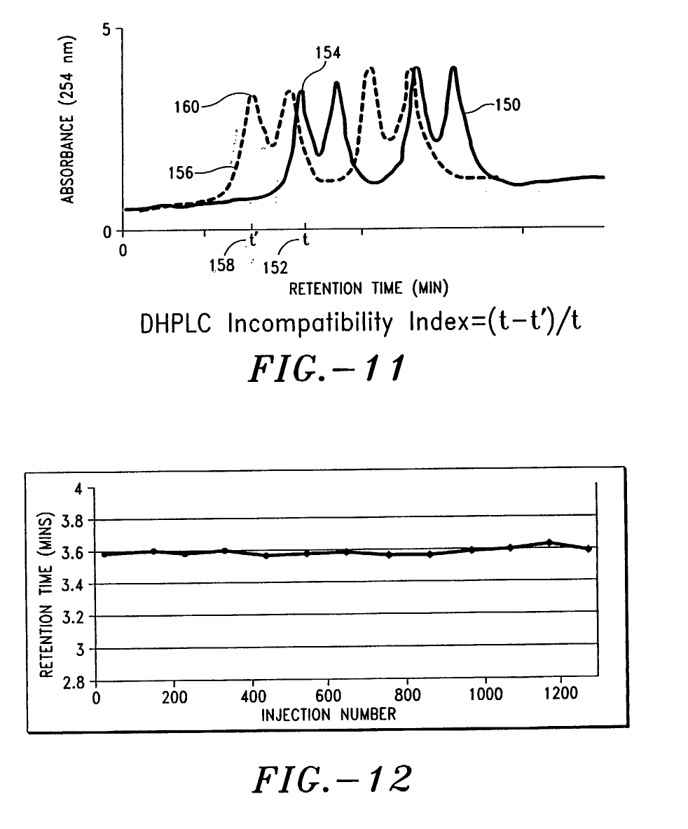

As mentioned in reference to FIGS. 9 and 10, Applicants have found that some PCR buffers interfere with DHPLC analysis and exhibit a DHPLC Incompatibility Index of about 0.1 or more. In some cases, it was possible to recover the performance of the separation column by including a washing procedure. However, this takes additional time, and the results prior to the washing procedure were adversely affected. In some cases, the performance of the separation column could not be recovered.

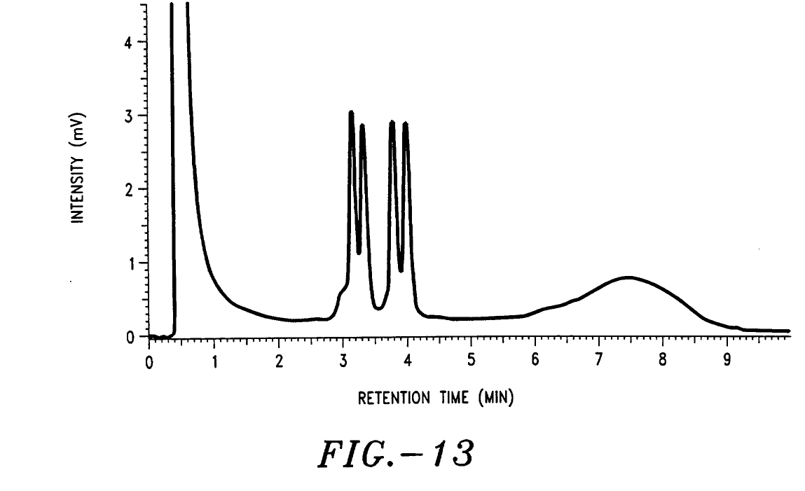

Using the above Index, Applicants have devised PCR buffers that are compatible with mutation detection by DHPLC analysis. In one embodiment, a PCR buffer at its working concentration (i.e. 1×) includes one or more non-ionic detergents at a concentration in the range of about 0.001% to about 0.01% volume/total volume of buffer. Preferably, the concentration is less than or equal to about 0.01%. The concentration of the non-ionic detergent in the PCR buffer is operably less than about 1% (volume/volume of buffer), preferably no greater than about 0.095%, more preferably no greater than about 0.05%, and most preferably no greater than about 0.01%. The non-ionic detergent can be present in the range of about 0.05% to about 0.001%, and preferably in the range of about 0.02% to about 0.001%. The PCR buffer can include salts, buffering agent, magnesium, and other compounds as indicated hereinabove. An example of a suitable PCR buffer (1×) is as follows: KCl (75 mM), Tris (pH 8.8, 10 mM), MgSO₄ (1.5 mM), Triton X-100 (0.01%). FIG. 12 illustrates the effect of multiple injections of this PCR buffer on the performance of a separation column as measured by the retention time of heteroduplex DNA in the 209 bp Mutation Standard mixture of homoduplex and heteroduplex molecules.

Using the above Index, a variety of components that are routinely used in PCR mixtures have been found to interfere with analysis by DHPLC. These include the following: bovine, equine, rat, chicken, goat, or baboon serum albumin; metal ions; mineral oil; formamide; and particulate matter. Preferred PCR buffers are substantially free of, and more preferably are devoid of, these interfering agents.

Using the above Index, Applicants have also devised PCR buffers that are preferably devoid of, or which contain minimal concentrations of, components that can interfere with DHPLC analysis. Such inhibitors can include one or more of the following: unidentified “proprietary” ingredients such as “stabilizers”, “enhancers” or “additives”; Bovine serum albumin (BSA); autoclaved water; mineral oil; formamide; Proteinase K; high molecular weight stabilizers such as polyethylene glycol (PEG); detergents such as Triton X-100, NP40, Tween 20, sodium dodecyl sulfate; sodium lauryl sulfate. Other reagents, such as those commonly used in the purification of DNA, such as proteases, solvents, nucleases, phenol, guanidinium, etc., are inhibitors of DNA polymerase activity and also may show incompatibility with the reverse phase column. If these reagents are used, it is preferred to carry out a final ethanol precipitation and wash step to remove most of these contaminants prior to PCR. Excess EDTA, isopropanol, or iso-amyl alcohol can inhibit the PCR, and are preferably removed prior to PCR.

Certain compounds may be present in the PCR mixture, but preferably do not exceed concentrations (as shown in parentheses) that minimize interference with DHPLC analysis: glycerol (2%), DMSO (10%), betaine (1.25-2.5 M).

The DHPLC Incompatibility Index can be used to devise other PCR solutions, such as storage buffers for DNA polymerases. There are a variety of different DNA polymerase enzymes that can be used in this aspect of the invention, although proofreading polymerases are preferred. DNA polymerases useful in the present invention may be any polymerase capable of replicating a DNA molecule. Preferred DNA polymerases are thermostable polymerases, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof. Other DNA polymerases are known in the art and can also be in the instant invention. Preferably the thermostable DNA polymerase is selected from the group of Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, PFUTurbo™, AmpliTaq®, AccuType™, or mixtures thereof, and active mutants, variants and derivatives thereof. It is to be understood that a variety of DNA polymerases may be used in certain aspects of the present invention, including DNA polymerases not specifically disclosed above, without departing from the scope or preferred embodiments thereof.

Solutions for storing DNA polymerases can include one or more of the following components: buffering agents (e.g. Tris-HCI, HEPES), metal chelating agents (e.g. ethylenediamine tetraacetic acid (EDTA)), reducing agents (e.g. β-mercaptoethanol, dithiothreitol), non-ionic detergent (e.g. Triton X-100), gelatin, an ethoxylated nonyl phenol, a polyoxyethylated sorbitan monolaurate and glycerol, for example.

In yet another aspect, the present invention encompasses kits for use in detecting mutations in a double stranded DNA fragment. The kits may comprise one or more of the following: instructional material; a container that contains Pho DNA polymerase; Pho DNA polymerase in a storage solution, wherein said storage solution is preferably characterized by having a DHPLC Incompatibility Index of no greater than 0.05; one or more PCR primers; Pho DNA polymerase in a storage solution, wherein said storage solution comprises a non-ionic detergent, wherein said detergent is present at a concentration of no more than 0.1% volume/volume of solution and wherein said solution is devoid of BSA; a container which contains PCR buffer; a container which contains a PCR buffer wherein said buffer is characterized by having a DHPLC Incompatibility Index of no greater than 0.05; a container which contains a PCR buffer wherein said buffer comprises a non-ionic detergent, wherein said detergent is present at a concentration of no more than 0.01% volume/volume of said buffer when said buffer is present in a PCR mixture.

The kits can also contain one or more of a separation column (e.g. a reverse phase separation column or an ion exchange separation column) for use in separating DNA molecules; a liquid chromatography system; software for operating the chromatography system; software for analyzing data generated from the liquid chromatographic analysis of the DNA molecules; and software for analyzing and modeling the melting properties of DNA molecules (i.e. primer design software).

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patent applications, patents, and literature references cited in this specification are hereby incorporated by reference in their entirety. In case of conflict or inconsistency, the present description, including definitions, will control. Unless mentioned otherwise, the techniques employed or contemplated herein are standard methodologies well known to one of ordinary skill in the art. The materials, methods and examples are illustrative only and not limiting.

All numerical ranges in this specification are intended to be inclusive of their upper and lower limits.

Other features of the invention will become apparent in the course of the following descriptions of exemplary embodiments which are given for illustration of the invention and are not intended to be limiting thereof.

Procedures described in the past tense in the Examples below have been carried out in the laboratory. Procedures described in the present tense have not yet been carried out in the laboratory, and are constructively reduced to practice with the filing of this application.

### EXAMPLE 1

### Standard PCR Conditions

The following is an example of cycling conditions that can be used as a starting point for PCR reactions. The conditions assume a reaction volume of 50 μL and a target fragment of 500 base pairs. The number of cycles used for PCR is a balance between the yield required from the reaction and the need to preserve optimum reaction conditions. As PCR proceeds, the conditions in the reaction change because dNTPs are polymerized to form the PCR product. The polymerase is slowly denatured and the relative concentration of different components change.

In general, the absolute number of error incorporated in each cycle increases with increasing cycle number throughout the course of the PCR. Therefore it is preferable to use the minimum number of cycles required to achieve sufficient product yield. The example is for a typical Three-Step PCR reaction as well as Touchdown PCR. These methods are preferred as a starting point from which to optimize most reactions.

Tₐ is the annealing temperature. (Tₐ₌3° C. above the average of forward primer Tm and reverse primer T. Ideally the difference between the Tm values for the individual primers in a pair should not be more then 2° C.)

The Unit of activity for Pho polymerase is defined as follows: the amount of enzyme that will incorporate 10 nmoles of dNTPs into acid insoluble material per 30 minutes at 74° C. under defined reaction conditions.

The concentration of the Pho polymerase in the PCR mixture (i.e. the working concentration) is preferably in the range of 0.01 units per μL to 0.05 units per μL.

This example demonstrates the use of a reaction buffer (i.e. PCR buffer) of the present invention.

Reaction Mix:

Optimase™p olymerase (B) 0.5 to 1 μL (2.5 units)

Forward Primer(A) 0.4 to 0.6 μM final concentration

Reverse Primer(A) 0.4 to 0.6 μM final concentration

PCR buffer(C) 5 μL of a 10× stock solution

Template DNA(A) 100 to 150 ng (Human Genomic DNA)

dNTPs(A) 200, μM final concentration of each dNTP

MgSO₄(A) 1.5 mM final concentration

Water To 50 μL

Cycling conditions (Standard method):

Step 1 95° C. 5 minutes

Step 2 95° C. 30 seconds(B)

Step 3 Tₐ° C. (A) 30 seconds to 1 minute(B)

Step 4 72° C. (B) 1 minute per 500 base pairs(A)

Steps 2 to 4 repeat 25 to 30 times

Step 5 72° C. 5 minutes

Hold at 4° C.

Cycling conditions (Touchdown method):

Step 1 95° C. 5 minutes

Step 2 95° C. 30 seconds

Step3Tₐ+7° C. 30 seconds

Reduce temperature by 0.5° C. per cycle

Step 4 72° C.(B) 1 minute per 500 base pairs(A)

Steps 2 to 4 repeat 13 times

Step 5 95° C. 20 seconds(B)

Step 6 Tₐ° C.(A) 1 minute(B)

Step 7 72° C.(B) 1 minute per 500 base pairs(A)

Steps 5 to 7 repeated 19 times

Step 8 72° C. 5 minutes

Hold at 4° C.

### EXAMPLE 2

### Comparison of Fidelity of DNA Polymerases

PCR reactions were set up using Pho polymerase in parallel with six commercially available polymerases known to be in common use for the preparation of samples for DHPLC analysis. For each polymerase tested, except Pho, the concentration of primers, Mg²⁺, dNTPs, polymerase and all buffer components were used exactly as specified by the manufacturer.

The PCR buffer (1×) used with Pho polymerase contained the following components: KCl (75 mM), Tris (pH 8.8, 10 mM), MgSO₄ (1.5 mM), Triton X-100 (0.01%). Pho was maintained in a storage solution comprising: 40 mM Tris-HCl (pH 7.5), 0.1 mM EDTA, 5 mM β-mercaptoethanol, 0.1% (volume/total volume storage solution) Triton X-100, and 60% (volume/total volume storage solution) glycerol.

Briefly, a 500 bp fragment was amplified in a reaction consisting of 2.5 units of polymerase, 1 μM of each primer, PCR buffer and Mg²⁺ to the manufacturers recommendations, approximately 1×10⁵ copies of λDNA template, 200 μM of each dNTP and water to a final volume of 50 μL. Cycling conditions were as shown in TABLE 3, with a hot start step included as recommended by the manufacturer for those enzymes requiring this procedure.

TABLE 3

| Step | Temperature | Duration |
|---|---|---|
| Initial denaturation | 95° C. | 2 min |
| 13 cycles of: | | |
| 1 | 94° C. | 20 sec |
| 2 | 65° C. (less than 0.5° C. per cycle) | 1 min |
| 3 | 72° C. | 1 min |
| 19 cycles of: | | |
| 4 | 94° C. | 20 sec |
| 5 | 56° C. | 1 min |
| 6 | 72° C. | 1 min |
| Final extension | 72° C. | 5 min |

Products were then hybridized to ensure representative heteroduplex formation by heating at 95° C. for 10 minutes followed by decreasing the temperature at a rate of 1.5° C. per minute until a final temperature of 25° C. was reached.

Each PCR product was analyzed using DHPLC at a predicted optimum temperature of 62° C. with a flow rate of 0.9 mL/min and 10 μL injection volume. The separation column (50×4.6 mm ID) was a DNASep® column (Transgenomic). A solvent gradient was generated by mixing eluent A (0.1 M TEAA pH 7.0) and B (0.1 M TEAA, 25% acetonitrile, pH 7.0) in a linear gradient running from 59 to 67% eluent B over 4 minutes. Following each analytical run the reverse phase column was washed using 100% buffer B for 0.5 minutes and then equilibrated at 54% B for 2 minutes in preparation for the next sample injection. Peak areas for homoduplex and heteroduplex peaks were calculated to allow determination of the percentage of PCR fragments forming heteroduplex DNA, indicating the presence of PCR induced errors. Assays were carried out at least in triplicate at three separate locations to ensure data were representative of different working practices and equipment.

TABLE 3 shows cycling conditions used in PCR amplification of test fragments and follows the procedures described by Cline et al. (Nucleic Acids Research 24:3546-3551 (1996)). The addition of a hot start procedure (10 minutes at 95° C.) was applied where recommended by the polymerase manufacturer.

In FIG. 2, a 500-bp fragment was amplified from λ phage genomic DNA with various polymerases with and without proofreading activity. Pho polymerase (Transgenomic's Optimase Polymerase) with proofreading activity was one of the polymerases tested. Pho polymerase results are shown in the bottom chromatogram. Due to the high yield obtained with Pho, chromatograms of PCR products for this polymerase were scaled by a factor of 0.3. PCR buffers used in PCR were those provided with each of the commercial polymerases tested. All amplifications were performed under identical cycling conditions. The fidelity of polymerization was assessed at 62° C., which is the software predicted temperature for DHPLC of the amplified DNA fragment. Errors caused by polymerase infidelity led to the formation of many different heteroduplexes. Heteroduplexes eluted earlier than homoduplexes and were apparent as broad peaks preceding the homoduplex peak. As shown, all polymerases except Pho showed significant error incorporation that could interfere with mutation detection. Heterodulexes formed due to the presence of sequence variations in the template will co-elute with those heteroduplexes formed due to polymerase-induced errors. As the fidelity of the polymerases decreases, formation of heteroduplexes resulting from PCR products carrying polymerase-induced errors will increase. As a consequence, accurate and reliable identification of true sequence variations will become increasingly difficult.

### EXAMPLE 3

### Amplification and DHPLC Analyses of Ras Alleles

Genomic DNA was isolated from cell lines possessing previously characterized G12D and G13DD ras alleles. All amplifications applied “Platinum Taq”, and the concentration of primers, Mg²⁺, dNTPs, polymerase and all buffer components were used exactly as specified by the manufacturer. In addition to these conditions, the ras amplifications were performed in the presence of 6% DMSO. The amplifications used a 6-FAM-labeled, PAGE-purified forward primer (CGCCCGCCGCCGCCCGCCGCCCGTCCCGCCATATAGTCACATTTTCATT ATTTTTATTATAAGG (SEQ ID NO: 1), non-template GC-clamp sequence italicized) and an unlabeled PAGE-purified reverse primer (AATTAGCTGTATCGTCAAGGCACTC) (SEQ ID NO: 2). Amplifications were performed by heat denaturation at 94° C. for 1 minute, followed by 35 cycles of: 94° C. for 15 seconds, 56° C. for 15 seconds, 70° C. for 15 seconds. Upon completion, a separate hybridization reaction was performed by heating to 95° C. for three minutes, followed by cooling at −0.1° C./second to 25° C.

The amplified ras alleles were analyzed by injecting 10 μL of PCR product into the Wave® Fragment Analysis System. Fragment detection was achieved by tuning the fluorescence detector to 496 rim excitation/520 nm emission. Chromatographic eluent “A” was 0.1 M triethylammonium acetate, and eluent “B” was 0.1 M triethylammonium acetate, 25% (v/v) acetonitrile. The gradient is shown in TABLE 4, with a column temperature of 59° C. The end of each run was subjected to an automated column regeneration/clean-off with 500 μL of 75% (v/v) acetonitrile.

TABLE 4

| Time | % A | % B |
|---|---|---|
| 0 | 60 | 40 |
| 0.1 | 55 | 45 |
| 12.1 | 43 | 57 |
| 14.5 | 60 | 40 |

### EXAMPLE 4

### Amplification and DHPLC Analyses of pBR322 Amplicons

2 ng of pBR322 plasmid were used for the amplifications. Taq, Pfu and Pho polymerases were used for the pBR322 amplifications. The concentration of primers, Mg²⁺, dNTPs, polymerase and all buffer components were used exactly as specified by the manufacturers. All of the amplifications used an unlabeled PAGE-purified forward primer (CGCCCGCCGCCGCCCGCCGCCCGTCCCGCCGCTCATCGTCATCCTCGG CA (SEQ ID. NO: 3), non-template GC-clamp sequence italicized) and an unlabeled PAGE-purified reverse primer (AAGTAGCGAAGCGAGCAGGACTGG) (SEQ ID. NO: 4). Amplifications were performed by heat denaturation at 95° C. for 3 minutes, followed by 25 cycles of: 95° C. for 1 minute, 57° C. for 1 minute, 72° C. for 1 minute. A final extension step at 72° C. was performed for 10 minutes. Upon completion, a hybridization step was performed by heating the products to 95° C. for three minutes, followed by cooling at −0.1° C./second to 25° C. Amplifications performed with Taq polymerase were further treated with 2 units of Klenow fragment for 15 minutes at 30° C., followed by inactivation of the Klenow fragment with 5 μL of 0.5 M EDTA. This extra step ensured that any Taq-derived dATP overhangs were eliminated.

The pBR322 amplification products were analyzed by injecting 10 μL of PCR product into the Wave® Fragment Analysis System. Fragment detection was achieved by tuning the UV absorbance detector to 260 nm. Chromatographic eluent “A” was 0.1 M triethylammonium acetate, and eluent “B” was 0.1 M triethylammonium acetate, 25% (v/v) acetonitrile. The gradient is shown in TABLE 5, with a column temperature of 65° C. The end of each run was subjected to an automated column regeneration/clean-off with 500 μL of 75% (v/v) acetonitrile.

TABLE 5

| Time | % A | % B |
|---|---|---|
| 0 | 56 | 44 |
| 0.1 | 51 | 49 |
| 10.1 | 41 | 59 |
| 12.5 | 56 | 44 |

### EXAMPLE 5

### Determination of DHPLC Incompatibility Index

The DNA fragment used in the determination of the DHPLC Incompatibility Index comprises a mutant and a wild type 209-bp fragment. Upon hybridization, the mixture includes homoduplex and heteroduplex dsDNA as shown schematically in FIG. 1

The 209 bp Mutation Standard contains equal amounts of the double stranded sequence variants 168A and 168G of the 209 base pair fragment from the human Y chromosome locus DYS271 (GenBank accession Number S76940). The A→G transition position 168 in the sequence was reported by Seielstad et al. (Human Molecular Genetics 3:2159-2161 (1994)) and the preparation of the variants has been described (Narayanaswami et al, Genetic Testing 5:9-16 (2001)).

The following is the sequence of the 168G variant:

(SEQ ID NO: 5)

AGGCACTGGTCAGAATGAAGTGAATGGCACACAGGACAAGTCCAGACCCA
GGAAGGTCCAGTAACATGGGAGAAGAACGGAAGGAGTTCTAAAATTCAGG
GCTCCCTTGGGCTCCCCTGTTTAAAAATGTAGGTTTTATTATTATATTTC
ATTGTTAACAAAAGTCCA_TGAGATCTGTGGAGGATAAAGGGGGAGCTGT
ATTTTCCATT

The following is the sequence of the 168A variant:

(SEQ ID NO: 6)

AGGCACTGGTCAGAATGAAGTGAATGGCACACAGGACAAGTCCAGACCCA
GGAAGGTCCAGTAACATGGGAGAAGAACGGAAGGAGTTCTAAAATTCAGG
GCTCCCTTGGGCTCCCCTGTTTAAAAATGTAGGTTTTATTATTATATTTC
ATTGTTAACAAAAGTCCG_TGAGATCTGTGGAGGATAAAGGGGGAGCTGT
ATTTTCCATT

In the Mutation Standard, the fragments are present at a total DNA concentration of 45 μg/mL and suspended in 10 mM Tris-HCl, pH 8, 1 mM EDTA.

This Mutation Standard is available commercially from Transgenomic (WAVE® System Low Range Mutation Standard, part no. 560077) and a similar standard is available from Varian (Walnut Creek, Calif.).

Prior to analysis, the Mutation Standard is hybridized by heating to 95° C. for 12 min, then cooled to 25° C. for 30 min.

The chromatography system is the WAVES DNA Fragment Analysis system (Transgenomic). The separation column is a 50×4.6 mm ID DNASep® column (Transgenomic) containing alkylated poly(styrene-divinylbenzene) beads.

Eluents used for the separation are: Buffer A, 0.1 M triethylammonium acetate (TEAA), pH 7.0 (Transgenomic) in water; Buffer B, 0.1 M TEAA and 25% acetonitrile in water pH 7.0. The elution of DNA fragments is monitored with a UV detector at 254 nm. The flow rate is 0.9 mL/min. The mobile phase gradient is as follows:

| Time | A % | B % |
|---|---|---|
| 0.0 | 50 | 50 |
| 0.5 | 47 | 53 |
| 4.0 | 40 | 60 |
| 5.0 | 0 | 100 |
| 6.5 | 50 | 50 |
| 8.5 | 50 | 50 |

A volume of 5 μL Mutation Standard is injected onto the separation column and eluted at 56° C., a temperature which partially denatures at a site of mismatch and a chromatogram is recorded. The resulting chromatogram is shown in FIG. 13. The retention time of the earliest eluting heteroduplex peak in the chromatograph is obtained, and if necessary, conditions are adjusted so that this retention time is about 3.3 min.

Subsequently, 5 μL of the PCR buffer (storage buffer or other solution) being tested is injected onto the separation column and eluted under the same conditions. This injection and elution is repeated 100 times and simulates the routine analysis of a PCR mixture.

Prior to injection, any PCR buffer, storage solution, or other solution being characterized is diluted to its “working concentration”. The working concentration is the concentration that would be present in a PCR mixture during an actual PCR. An example of a PCR mixture is provided in the “Reaction Mix” in EXAMPLE 1. For example, a PCR buffer is often provided, such as in a kit, as a 10-fold concentrated solution to be combined with DNA polymerase, template, NTPs, and other components. In the instant method, such a PCR buffer is diluted by a factor of 10, with double distilled water, prior to injection, in order to simulate actual concentrations present during PCR.

After the 100 injections, the column is again tested by injecting the Mutation Standard. From the chromatogram 156, the retention time 158 of the earliest eluting heteroduplex peak 160 is determined (FIG. 11).

The DHPLC Incompatibility Index is calculated according to the following equation:

DHPLC Incompatibility Index=(t−t′)/t

where t is the retention time 152 of the first eluting heteroduplex peak prior to the 100 injections of PCR buffer, and where t′ is the retention time 158 of the first eluting heteroduplex peak after the 100 injections of PCR buffer.

### EXAMPLE 6

### Determination of a DHPLC Mutation Index for a Storage Solution for Pho Polymerase

A storage solution for Pho polymerase was prepared which includes the following components in a 10× solution: 40 mM Tris HCl (pH 7.5), 0.1 mM EDTA, 5 mM β-mercaptoethanol, 0.1% (volume/volume total storage solution) Triton X-100, and 60% (volume/total volume storage solution) glycerol. The DHPLC Incompatibility Index of the storage solution is determined after a ten-fold dilution in water and is found to be less than 0.05.

### EXAMPLE 6

### Determination of a DHPLC Mutation Index for a PCR Buffer

A PCR buffer (1×) is prepared as follows: KCl (75 mM), Tris (pH 8.8, 10 mM), MgSO₄ (1.5 mM), Triton X-100 (0.01% volume/total volume of buffer). The DHPLC Incompatibility Index of the PCR buffer is determined and is found to be less than 0.02.

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein.

All patent applications, patents, and literature references cited in this specification are hereby incorporated by reference in their entirety. In case of conflict or inconsistency, the present description, including definitions, will control.

The invention claimed is:

1. A method for mutation detection of a double stranded DNA fragment by denaturing high performance liquid chromatography, the double stranded DNA fragment corresponding to a wild type double stranded DNA fragment having a known nucleotide sequence, the method comprising:
(a) amplifying a section of said double stranded DNA fragment by PCR using a set of primers which flank the ends of said section, wherein said PCR is conducted with Pho DNA polymerase;
(b) hybridizing the amplification product of step (a) with wild type double stranded DNA corresponding to said section, whereby a mixture comprising one or more heteroduplexes is formed if said section includes a mutation; and
(c) analyzing the product of step (b) by denaturing high performance liquid chromatography.

2. The method of claim 1 wherein the section being amplified is indicative of a disease state.

3. A method for mutation detection of a double stranded DNA fragment by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with a proofreading DNA polymerase;
(b) hybridizing the amplification product of step (a) with wild type double stranded DNA corresponding to said section, whereby a mixture comprising one or more heteroduplexes is formed if said section includes a mutation; and
(c) analyzing the product of step (b) by denaturing high performance liquid chromatography,
wherein said PCR is conducted in a PCR buffer, wherein said PCR buffer is characterized by having a DHPLC Incompatibility Index no greater than 0.05.

4. The method of claim 3 wherein said PCR buffer comprises one or more non-ionic detergents having a total concentration no greater than 0.01% volume/total volume of PCR buffer.

5. A method for mutation detection of a double stranded DNA fragment by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with a proofreading DNA polymerase;
(b) hybridizing the amplification product of step (a) with wild type double stranded DNA corresponding to said section, whereby a mixture comprising one or more heteroduplexes is formed if said section includes a mutation; and
(c) analyzing the product of step (b) by denaturing high performance liquid chromatography,
wherein said PCR mixture comprises a non-ionic detergent having a concentration no greater than 0.01% volume/total volume of the total PCR mixture.

6. The method of claim 5 wherein said mixture is characterized by having a DHPLC Incompatibility Index no greater than 0.05.

7. The method of claim 5 wherein said detergent comprises t-octylphenoxypolyethoxyethanol.

8. The method of claim 5 wherein said polymerase comprises Pho polymerase.

9. The method of claim 6 wherein said polymerase comprises Pho polymerase.

10. The method of claim 7 wherein said polymerase comprises Pho polymerase.

11. The method of claim 5 wherein said proofreading DNA polymerase is Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT, DEEPVENT, PFUTurbo, AmpliTaq, or a combination thereof.

12. The method of claim 11 wherein said polymerase is an active mutant, variant or derivative of a proofreading DNA polymerase.

13. A method for mutation detection of a double stranded DNA fragment by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with Pho DNA polymerase;
(b) hybridizing the amplification product of step (a) with wild type double stranded DNA corresponding to said section, whereby a mixture comprising one or more heteroduplexes is formed if said section includes a mutation; and
(c) analyzing the product of step (b) by denaturing high performance liquid chromatography,
wherein said PCR is conducted in a PCR buffer, wherein said PCR buffer is characterized by having a DHPLC Incompatibility Index no greater than 0.05.

14. A method for mutation detection of a double stranded DNA fragment by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with Pho DNA polymerase;
(b) hybridizing the amplification product of step (a) with wild type double stranded DNA corresponding to said section, whereby a mixture comprising one or more heteroduplexes is formed if said section includes a mutation; and
(c) analyzing the product of step (b) by denaturing high performance liquid chromatography,
wherein said PCR is conducted in a PCR buffer, wherein said PCR buffer comprises a non-ionic detergent having a concentration no greater than 0.01% volume/total volume of said PCR buffer.

15. A method for mutation detection of a double stranded DNA fragment by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with Pho DNA polymerase;
(b) hybridizing the amplification product of step (a) with wild type double stranded DNA corresponding to said section, whereby a mixture comprising one or more heteroduplexes is formed if said section includes a mutation; and
(c) analyzing the product of step (b) by denaturing high performance liquid chromatography wherein prior to step (a), said polymerase is stored in a storage solution, wherein a portion of said storage solution is included with said PCR mixture,
wherein said PCR mixture is characterized by having a DHPLC Incompatibility Index no greater than 0.05.

16. The method of claim 15 wherein said PCR is conducted in a PCR buffer, wherein said PCR buffer comprises a non-ionic detergent having a concentration no greater than 0.01%.

17. The method of claim 16 wherein said storage solution comprises t-octylphenoxypolyethoxyethanol at a concentration no greater than 0.01%.

18. A composition for use in preparing samples for analysis by denaturing high performance liquid chromatography, said composition comprising:
Pho DNA polymerase, and
one or more non-ionic detergents present in a concentration no greater than 0.01% volume/total volume of said composition.

19. The composition of claim 18 wherein said composition is devoid of bovine serum albumin.

20. The composition of claim 18 wherein said detergent comprises t-octylphenoxypolyethoxyethanol.

21. A composition for use in preparing samples for analysis by denaturing high performance liquid chromatography, said composition comprising:
Pho DNA polymerase, and
non-ionic detergent present in a concentration no greater than 0.1% volume/total volume of said composition,
wherein said composition is characterized by a DHPLC Incompatibility Index of no greater than 0.05.

22. A composition for use in preparing samples for analysis by denaturing high performance liquid chromatography, said composition comprising:
a proofreading DNA polymerase, and
non-ionic detergent present in a concentration no greater than 0.1% volume/total volume of said composition,
wherein said composition is characterized by a DHPLC Incompatibility Index of no greater than 0.05.

23. The composition of claim 22 wherein said proofreading DNA polymerase is Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT, DEEPVENT, PFUTurbo, AmpliTaq or a mixture thereof.

24. The composition of claim 23 wherein said polymerase is an active mutant, variant or derivative of a proofreading DNA polymerase.

25. A composition for use in preparing samples for analysis by denaturing high performance liquid chromatography, said composition comprising:
Pho DNA polymerase,
non-ionic detergent present in a concentration no greater than 0.01%,
and wherein said composition is devoid of serum albumin.

26. The composition of claim 25 wherein said serum albumin comprises bovine serum albumin.

27. The composition of claim 25 wherein said composition is characterized by having a DHPLC Incompatibility Index no greater than 0.5.

28. A composition for use in preparing samples for analysis by denaturing high performance liquid chromatography, said composition comprising:
Pho DNA polymerase,
wherein said polymerase is stored in a storage solution,
wherein a portion of said storage solution is included in a PCR mixture which also includes a PCR buffer,
wherein said PCR mixture is characterized by a DHPLC Incompatibility Index of no greater than 0.05.

29. The composition of claim 28 wherein said storage solution comprises t-octylphenoxypolyethoxyethanol at a concentration no greater than 0.5% volume/total volume of said composition.

30. The composition of claim 28 wherein said storage solution comprises t-octylphenoxypolyethoxyethanol at a concentration no greater than 0.1%.

31. A composition for use in preparing samples for analysis by denaturing high performance liquid chromatography, said composition comprising:
a proofreading polymerase,
wherein said polymerase is stored in a storage solution,
wherein when said storage solution is included in a PCR mixture,
wherein said PCR mixture is characterized by having a DHPLC Incompatibility Index no greater than 0.05.

32. A composition for use in preparing samples for analysis by denaturing high performance liquid chromatography, said composition comprising:
a proofreading polymerase,
wherein said polymerase is stored in a storage solution,
wherein when said storage solution is characterized by having a DHPLC Incompatibility Index no greater than 0.01.

33. A kit for preparing a double stranded DNA for mutation detection by denaturing high performance liquid chromatography, said kit comprising:
(a) a container which contains a composition comprising Pho polymerase, and which contains non-ionic detergent present at a concentration no greater than 0.1%,
wherein said composition is devoid of bovine serum albumin, and
(b) a container which contains a mutation standard.

34. The kit of claim 33 wherein said detergent comprises t-octylphenoxypolyethoxyethanol.

35. The kit of claim 33 further comprising a container which contains one or more PCR primers.

36. The kit of claim 33 further comprising a separation column for use in denaturing high performance liquid chromatography.

37. A kit for preparing a double stranded DNA for mutation detection by denaturing high performance liquid chromatography, said kit comprising:
(a) a container which contains a composition comprising Pho DNA polymerase, and which contains non-ionic detergent present in a concentration no greater than 0.1%,
(b) a container which contains a PCR buffer,
wherein said PCR buffer is characterized by having a DHPLC Incompatibility Index no greater than 0.05.

38. A kit for preparing a double stranded DNA for mutation detection by denaturing high performance liquid chromatography, said kit comprising:
(a) a container which contains a composition comprising Pho DNA polymerase, and
(b) a container which contains a PCR buffer,
wherein said PCR buffer contains non-ionic detergent present in a concentration no greater than 0.01% volume/total volume of said buffer.

39. The kit of claim 38 wherein said detergent comprises t-octylphenoxypolyethoxyethanol.

40. A kit for preparing a double stranded DNA for mutation detection by denaturing high performance liquid chromatography, said kit comprising:
(a) a container which contains a composition comprising a proofreading DNA polymerase,
(b) a container which contains a PCR buffer,
wherein said PCR buffer is characterized by having a DHPLC Incompatibility Index no greater than 0.05.

41. The kit of claim 40 wherein said polymerase comprises Pho polymerase.

42. The kit of claim 40 wherein said proofreading DNA polymerase is Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT, DEEPVENT, PFUTurbo, AmpliTaq, or a mixture thereof.

43. The kit of claim 42 wherein said polymerase is an active mutant, variant or derivative of a proofreading DNA polymerase.

44. The kit of claim 40 wherein said composition comprising a proofreading DNA polymerase further comprises one or more non-ionic detergents present in a total concentration no greater than 0.1% volume/total volume of said composition, and wherein said composition is devoid of serum albumin.

45. A kit for use in preparing samples for analysis by denaturing high performance liquid chromatography, said composition comprising:
(a) a container which contains a proofreading polymerase,
wherein said polymerase is stored in a storage solution,
wherein when said storage solution is included in a PCR mixture, said PCR mixture is characterized by having a DHPLC Incompatibility Index no greater than 0.05.

46. A kit for preparing a double stranded DNA for mutation detection by denaturing high performance liquid chromatography, said kit comprising:
(a) a container which contains a composition comprising a proofreading DNA polymerase,
(b) a container which contains a PCR buffer,
wherein said PCR buffer is characterized by having a DHPLC Incompatibility Index no greater than 0.1,
and wherein said buffer is devoid of bovine serum albumin.

47. A kit for preparing a double stranded DNA for mutation detection by denaturing high performance liquid chromatography, said kit comprising:
(a) a container which contains a composition comprising a proofreading DNA polymerase,
(b) a container which contains a PCR buffer,
wherein said PCR buffer is characterized by having a DHPLC Incompatibility Index no greater than 0.05,
wherein said buffer comprises KCl, Tris, MgSO₄, and wherein said buffer includes one or more non-ionic detergents at a concentration no greater than 0.01% volume/total volume of said buffer.

48. A kit for preparing a double stranded DNA for mutation detection by denaturing high performance liquid chromatography, said kit comprising:
(a) a container which contains a composition comprising a proofreading DNA polymerase,
(b) a container which contains a PCR buffer,
wherein said PCR buffer is characterized by having a DHPLC Incompatibility Index no greater than 0.05,
wherein said buffer comprises KCl (75 mM), Tris (pH 8.8, 1 mM), MgSO₄ (1.5 mM), and non-ionic detergent at a concentration of 0.01% volume/total volume of said buffer.

49. A method for preparing a sample of double stranded DNA fragment for mutation detection by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with a proofreading DNA polymerase;
(b) hybridizing the amplification product of step (a) with wild type double stranded DNA corresponding to said section, whereby a mixture comprising one or more heteroduplexes is formed if said section includes a mutation,
wherein said PCR is conducted in a PCR buffer, wherein said PCR buffer is characterized by having a DHPLC Incompatibility Index no greater than 0.05.

50. A method for preparing a sample of double stranded DNA fragment for mutation detection by denaturing high performance liquid chromatography, the double stranded DNA fragment corresponding to a wild type double stranded DNA fragment having a known nucleotide sequence, the method comprising:
(a) in a PCR mixture, amplifying a section of said double stranded DNA fragment by PCR using a set of primers which flank the ends of said section, wherein said PCR is conducted with Pho DNA polymerase;
(b) hybridizing the amplification product of step (a) with wild type double stranded DNA corresponding to said section, whereby a mixture comprising one or more heteroduplexes is formed if said section includes a mutation,
wherein said PCR is conducted in a PCR buffer, wherein said PCR buffer is characterized by having a DHPLC Incompatibility Index no greater than 0.05.

51. A method for preparing a sample of double stranded DNA fragment for mutation detection by denaturing high performance liquid chromatography, the double stranded DNA fragment corresponding to a wild type double stranded DNA fragment having a known nucleotide sequence, the method comprising:
in a PCR mixture, amplifying a section of said double stranded DNA fragment by PCR using a set of primers which flank the ends of said section, wherein said PCR is conducted with Pho DNA polymerase,
wherein said PCR is conducted in a PCR buffer,
wherein said PCR buffer is characterized by having a DHPLC Incompatibility Index no greater than 0.01.

52. A PCR buffer composition for use in preparing samples for analysis by denaturing high performance liquid chromatography, said composition comprising:
one or more non-ionic detergents present in a concentration no greater than 0.01% volume/total volume of said composition,
wherein said composition is characterized by having a DHPLC Incompatibility Index no greater than 0.01.
